(19) United States
(12) Patent Application Publication
(10) Pub. No.: US 2026/0327298 A1
(43) Pub. Date: Oct. 22, 2026

(54) BISPECIFIC ANTIBODIES FOR CANCER THERAPY

(71) Applicant: The University of North Carolina at Chapel Hill, Chapel Hill, NC (US)

(72) Inventors: Samuel Lai, Chapel Hill, NC (US); Alison M. Schaefer, Chapel Hill, NC (US); Limei SHEN, Chapel Hill, NC (US); Karthik TIRUTHANI, Chapel Hill, NC (US); Justin HUCKABY, Chapel Hill, NC (US)

(21) Appl. No.: 19/483,845

(22) PCT Filed: May 15, 2024
(86) PCT No.: PCT/US2024/029461

§ 371 Date: Nov. 12, 2025

Related U.S. Application Data
(60) Provisional application No. 63/466,518, filed on May 15, 2023.

(51) Int. Cl. C07K 16/22; A61K 39/00; A61P 35/04; C07K 16/28

(52) U.S. Cl. CPC C07K 16/22; A61P 35/04; C07K 16/2809; A61K 2039/505; C07K 2317/31; C07K 2317/522; C07K 2317/55; C07K 2317/565; C07K 2317/622; C07K 2317/73; C07K 2317/76; C07K 2317/92

(57) ABSTRACT

The present disclosure provides methods, compositions, systems, or kits for treating or delaying the progression of cancer. Particularly, the disclosure relates to therapeutic bispecific binding molecules (e.g., bispecific antibodies) that target cancer cell membrane components and anti-inflammatory or tumorigenic cytokines or chemokines or T cell activators.

### FIELD

The present disclosure relates to methods, compositions, systems and kits for treating or delaying the progression of cancer. Particularly, the disclosure relates to therapeutic bispecific binding molecules that target a cancer cell membrane component and one of an anti-inflammatory cytokine or chemokine, a tumorigenic cytokine or chemokine, or a T cell activator.

### CROSS-REFERENCE TO RELATED APPLICATIONS

This application claims the benefit of U.S. Provisional Application No. 63/466,518, filed May 15, 2023, the content of which is herein incorporated by reference in its entirety.

### SEQUENCE LISTING STATEMENT

The content of the electronic sequence listing titled UNC2_41053_601_SequenceListing.xml (Size: 12,930 bytes; and Date of Creation: May 13, 2024) is herein incorporated by reference in its entirety.

### BACKGROUND

Over the past decade, cancer immunotherapy has undergone a marked paradigm shift, from systemic activation of immune responses via immune enhancement to selective correction of defective immunity in the tumor microenvironment (TME) via immune normalization. Unfortunately, tumors can evade host immune surveillance through multiple distinct mechanisms, and it is also difficult to sustain effective concentrations of immunomodulators in the TME.

For example, the vast majority of triple-negative breast cancer (TNBC) patients failed to respond to monospecific immunomodulatory therapies in clinical trials, including those targeting PD-1/PD-L1 or CTLA4. The objective response rate, which measures the percentage of patients whose disease decreased (partial response) and/or disappeared (complete response) after treatment, is typically only ~20%. Furthermore, although combined immunotherapy and chemotherapy appeared promising in early trials, some of the observed efficacy was not reproduced in later clinical trials. For instance, Tencentriq failed to improve progression free survival and overall survival compared to paclitaxel alone, leading to its voluntary withdrawal in 2021. These developments strongly underscore the need of additional methods that could effectively normalize the immune TME in cancers.

### SUMMARY

Disclosed herein are methods of treating or delaying the progression of cancer in a subject in need thereof. In some embodiments, the methods comprise administering to a subject in need thereof an effective amount of a bispecific binding molecule configured to specifically bind: Siglec15 (S15) and one of: an anti-inflammatory cytokine or chemokine, a tumorigenic cytokine or chemokine, or a T cell activator.

Also disclosed herein are compositions for use in the disclosed methods. In some embodiments, the compositions comprise a bispecific binding molecule configured to specifically bind: Siglec15 (S15); and one of an anti-inflammatory cytokine or chemokine, a tumorigenic cytokine or chemokine, or a T cell activator for use in treating or delaying the progression of cancer in a subject.

In some embodiments, the anti-inflammatory chemokine or cytokine or tumorigenic chemokine or cytokine is selected from the group consisting of: TGF-β; IL-1 receptor agonist; IL-4; IL-10; IL-11; IL-13; IL-19; IL-20; IL-22; IL-23; IL-24; IL-26; IL-27; IL-28; IL-29; IL-33; IL-35; IL-37; IFN-α; IFN-γ; and CXCL12. In some embodiments, the anti-inflammatory chemokine or cytokine or tumorigenic chemokine or cytokine is TGF-β.

In some embodiments, the T cell activator is selected from the group consisting of: CD2, CD3, CD4, CD8, and CD28. In some embodiments, the T cell activator is CD3.

In some embodiments, the bispecific binding molecule is a bispecific antibody, or fragment thereof, having one or more antigen binding sites directed against S15 and one or more antigen binding sites directed against one of the anti-inflammatory cytokine or chemokine, the tumorigenic cytokine or chemokine, or the T cell activator.

In some embodiments, the bispecific antibody comprises one or more V_{H} domains each comprising an HCDR1, an HCDR2, and an HCDR3, and one or more V_{L} domains each comprising an LCDR1, an LCDR2, and an LCDR3 from an anti-Siglec15 antibody.

In some embodiments, the methods comprise administering two or more bispecific binding molecules. In some embodiments, a first bispecific binding molecule specifically binds Siglec15 (S15) and an anti-inflammatory cytokine or chemokine, a tumorigenic cytokine or chemokine. In some embodiments, the first bispecific binding molecule specifically binds Siglec15 (S15) and TGF-β. In some embodiments, a second bispecific binding molecule specifically binds Siglec15 (S15) and a T cell activator. In some embodiments, the second bispecific binding molecule specifically binds Siglec15 (S15) and CD3.

In some embodiments, the bispecific antibody comprises one or more antigen binding sites directed against the anti-inflammatory cytokine or chemokine, the tumorigenic cytokine or chemokine, or the T cell activator.

In some embodiments, the bispecific antibody comprises one or more V_{H} domains each comprising an HCDR1, an HCDR2, and an HCDR3, and one or more V_{L} domains each comprising an LCDR1, an LCDR2, and an LCDR3 from an antibody directed to an anti-inflammatory cytokine or chemokine, a tumorigenic cytokine or chemokine, or a T cell activator.

In some embodiments, the cancer is breast cancer, liver cancer, lung cancer, or pancreatic cancer. In some embodiments, the cancer is triple negative breast cancer. In some embodiments, the cancer is pancreatic cancer.

In some embodiments, administering reduces or clears the tumor burden in the subject, decreases or prevents metastasis, increases survival, or a combination thereof. In some embodiments, administering activates T cells. In some embodiments, administration of the bispecific binding molecule has increased effectiveness compared to methods comprising administration of an antibody against the cancer cell membrane-associated target and/or an antibody against the anti-inflammatory cytokine or chemokine, the tumorigenic cytokine or chemokine, or the T cell activator alone.

In some embodiments, the methods further comprise administering one or more chemotherapeutic agents to the subject, administering radiation therapy to the subject, surgical resection of cancerous tissue or tumors, or a combination thereof.

In some embodiments, the compositions further comprise one or more chemotherapeutic agents.

Other aspects and embodiments of the disclosure will be apparent in light of the following detailed description and accompanying figures.

### BRIEF DESCRIPTION OF THE DRAWINGS

FIGS. 1A-1D show expression and upregulation of Siglec15 (S15) and TGF-β in human PBMCs and breast cancer patient. PBMCs were harvested from different donor and analyzed by flow cytometry (FIG. 1A). Single cell staining and FMO were performed (n=3). S15 is upregulated in breast cancer tumor tissue compared to normal tissue, based on TCGA database (FIG. 1B). TGF-β was upregulated in different classes of breast cancer tumor tissue compared to normal tissue (FIG. 1C; TCGA data analysis, n=1100). Siglec15 expression was detected in triple negative breast cancer patient samples (FIG. 1D; n=3).

FIGS. 2A-2C show the characterization of a S15/TGF-β bispecific antibody (bsAb). FIG. 2A is a schematic of the structure of a Fab-IgG that binds ICAM⁻¹ and PEG. Orthogonal amino acid mutations, denoted using the Kabat numbering scheme, were introduced to ensure proper heavy- and light-chain pairings. FIG. 2B is a corresponding sequence map of the heavy chain and two light chains used to produce the Fab-IgG through simple co-expression. FIG. 2C is a graph of the binding affinity of S15/TGF-β bsAb to Siglec-15 compared to parent S15 mAb, measured by microScale thermophoresis.

FIGS. 3A-3C show detection of S15 expression in vitro on 4T1 tumor cells and binding of siglec15-TGF-β bsAb in mouse 4T1 triple negative breast tumor cells. 4T1 cells from cell culture were analyzed with anti-S15 mAb by flow cytometry (FIG. 3A). Tumors from mice inoculated with 4T1 cells orthotopically for 14 days were harvested, and single cell suspension were stained with ST bsAb. FMO were performed (FIG. 3B; n=5). Tumor tissues were harvested from mice with orthotopic 4T1 tumors, and stained for Siglec15 in Alex488 (FIG. 4C; red). Negative staining and control staining were performed.

FIGS. 4A-4E show that Siglec15-TGF-β3 bsAb (ST) effectively inhibited 4T1 tumor growth and markedly prolonged overall survival compared to mAb against S15 (S), TGF-β (T), and cocktail of both S15 and TGF-β (S+ T) mAbs. Tumor inhibition and host survival after ST bsAb in 4T1 triple negative breast cancer tumor models. The tumor-bearing mice were divided into 5 groups and treated with either control Ab, ST, S, T, S+ T. FIG. 4A is an exemplary timeline. FIGS. 4B and 4D are graphs of tumor growth inhibition in mice with orthotopic 4T1 tumors from two different rounds of experiments. Arrows indicate the time of treatment injection. FIGS. 4C and 4E are Kaplan-Meier survival curves of the variants treated groups in FIGS. 4B and 4D, respectively. Each group comprised of 5-8 mice (in FIG. 4B control: n=10, ST: n=8, S+ T: n=6, S: n=8, T:n=8; in FIG. 4E control: n=8,ST: n=10, S+ T: n=10, S: n=8,T:n=8;). Arrows indicate the days mice received IV injection of different mAbs. *p<0.05, **p<0.01 and ***p<0.001. The statistical analyses were calculated through comparison with the control group.

FIGS. 5A-5D shows that ST bsAb reduced immunosuppression within the TME. FIG. 5A are images showing ST treatment enhanced the presence of CD45+ cells while reducing the expression of S15 in TME. Tumor tissues were harvested at the end of the experiment. The sections were stained by CD45+ Alex594 (red) and Siglec15 Alex488 (green). Negative staining and control staining were performed. FIG. 5B is a graph showing that ST treatment increased CD45+ cells in tumor, as determined by flow cytometry of tumor-derived single cell suspension. Single staining and FMO were performed (n=5). FIG. 5C is graphs of the expression of various cytokines and chemokines confirmed more effective inhibition of immune suppression by ST vs. S, T, and S+ T treatment. Tumor tissue was harvested and homogenized with RIPI buffer. Tumor lysates were collected, and various cytokines/chemokines were quantified using Luminex. FIG. 5D shows that ST treatment increased activated CD4+ T-cells in the TME, assessed by flow cytometry of tumor-derived single cell suspension. Single staining and FMO were performed (n=5).

FIGS. 6A-6C show that ST bsAb treatment enhanced systemic anti-tumor T cell response. FIGS. 6A and 6B are flow cytometry and quantitation, respectively, showing increased T-cells in LN from ST-treated mice over vehicle control. Following bsAb treatment, increased activated CD4⁻ and CD8⁻ T-cells were detected in LN. LN were harvested, and single cell suspensions were isolated for flow cytometry (FIG. 6C). Single staining and FMO were performed. (n=5).

FIG. 7 shows images of the H&E staining of major organs in each group. Upper row are lung and lower row are the liver from tumor bearing mice from each group. The black arrows showed tumor metastasis.

FIG. 8 is a schematic of a potential mechanism of the S15/TGF-β3 BsAb. Untreated TNBC is an immunologically “cold” tumor with a highly immunosuppressive environment (Left). Upon treatment with a cocktail of S15 and TGF-β mAb, while S15 mAbs can bind TNBC targets and be preferentially retained, TGF-β3 mAb will not be preferentially retained within the tumor, and thus resulting in only limited TGF-β3 inhibition and modest reduction of the inherent immunosuppressive TME within TNBC (Middle). Treatment with S15/TGF-β biTMEM, due to S15 binding leading to greater extension of retention within the tumor, resulted in more complete inhibition of both S15 and TGF-β, and consequently the immunosuppressive TME (Right). This in turn transforms TNBC into an immunologically more active tumor, allowing immune cells to more effectively kill TNBC cells, prolong survival, and limit metastasis.

FIGS. 9A-9E show the therapeutic effect of siglec-15-CD3 BITE in human pancreatic cancer in NSG mice. FIGS. 9A and 9B are graphs of the change in tumor volume over time in each of the two rounds of treatment. FIG. 9C is an image of tumor size at experiment endpoint of Round 1 study. FIG. 9D is images of infiltration of human CD3 (red) in tumor tissues at the end of study. Left panel was tumor tissue from BITE treatment, middle panel was anti-Siglec15 mAb alone, and right panel was anti-CD3 mAb alone. Circulated CD3+CD45+ T cells in blood was evaluated by flow cytometry (FIG. 9E).

FIGS. 10A-10C show the design and characterization of an S15-CD3 BITE. FIG. 10A is a graph of the binding affinity as measured by ELISA. The left panel shows binding compared to parent molecule against S15, middle against CD3. The right panel shows the simultaneous binding affinity of the BITE against both targets. FIG. 10B is a schematic of the S15-CD3 BITE. FIG. 10C demonstrates the ability of the S15-CD3 BITE to promote T-cell proliferation in vitro, as assessed by CFSE proliferation assay. The histograms show a representative sample staining for T-cells after exposure to either control Ab or BITE. The bar graph represents the percentage of T-cells seen in each condition across multiple samples.

FIGS. 11A-11N show enhanced T cell killing with S15-CD3 BITE against siglec15+ human pancreatic cancer cell line (panc-1) and human lung cancer cell line(H460) cells. The remaining cells after 24-h co-culture of T cells and panc-1 or H460 cancer cells in presence or absence of STAB were analyzed by flow cytometry. FIGS. 11A-11F and 11M represent data in panc-1 cells, FIGS. 11G-11L and 11N represent data in H460 cells. FIGS. 11A and 11D demonstrate sample gating in the panc-1 cell line. FIGS. 11B and 11C show remaining CD3+CD45+ cell population in coculture after 72 hours incubation. FIGS. 11E and 11F show histograms of remaining panc-1 tumor cells in coculture after 72 hours of incubation. FIGS. 11G and 11J demonstrate sample gating in H460 cells. FIGS. 11H and 11I show remaining CD3+CD45+ cell population in coculture after 72 hours incubation. FIGS. 11K and 11L show histograms of remaining H460 tumor cells in coculture after 72 hours of incubation. FIGS. 11M and 11N show the percentage of cell death relative to control anti-human IgG at PBMC:Tumor cell ratios of 3:1 in panc-1 and H460 cell lines respectively.

FIGS. 12A-12G demonstrate experimental replication of S15-CD3 BITE ability to inhibit panc-1 tumor growth and prolong overall survival compared to αS15 and αCD3 mAB in vivo. FIG. 12A shows tumor growth inhibition in mice with subcutaneously panc-1 tumor model. FIG. 12B shows Kaplan-Meier survival curves of the variants treated groups, each comprised of 8-10 mice. FIG. 12C shows the tumor weight in each group at the experiment end point. FIGS. 12D-12G show levels of CD3+T cells and activated CD3+ T cells in blood 12 and 16 days after treatment.

FIGS. 13A-13H show that S15-CD3 BITE treatment increased T cell infiltration in tumor and reduced tumor associated fibroblast in desmoplastic panc-1 tumor. Tumors were harvested at the endpoint of the study. FIGS. 13A-13D show increased T-cell infiltration in sections stained by CD3+Alex594 (red) and Siglec15 Alex488 (green). FIGS. 13E-13G show reduced tumor-associated fibroblast infiltration in sections stained by αSMA⁺ Alex488 and DAPI in blue. FIG. 13H shows quantification of αSMA staining.

FIGS. 14A-14H show that that S15-CD3 BITE treatment increased T cell infiltration in tumor, as well as tumor inhibition and host survival after BITE and control Abs treatment in H460 lung tumor models in NSG mice. The tumor-bearing mice were treated either with PBMC and BITE or control antibody. FIG. 14A shows tumor growth inhibition in mice with subcutaneously injected H460 tumor model. FIG. 14B shows the Kaplan-Meier survival curves of each group, consisting of 8-10 mice. FIG. 14C shows the tumor weight at the experiment end point. FIGS. 14D and 14E show levels of CD3+ T cells activated CD3+ T cells in blood on days 12 and 16, respectively, after treatment.

FIGS. 14F-14H show staining for CD3+ T cells in tumor sections harvested at the study end-point. The sections were stained by CD3+ Alex594 (red) and DAPI in blue.

FIGS. 15A-15D show design and characterization of two different formats of S15/TGFβ bispecific antibodies. FIG. 15A shows schematics of the differing formats which were evaluated. ST IF is a format where the S15 Fab is on the N-terminus of the IgG and the TGFβ Fab is on the C-terminus. TS IF is a format where the TGFβ Fab is on the N-terminus and the S15 Fab on the C-terminus. FIG. 15B shows binding affinity compared to the parent IgG, demonstrating similar affinity for ST IF and TS IF, where TS IF is slightly reduced. FIG. 15C shows binding affinity against TGFβ. TS IF retains similar affinity compared to the parent IgG, while ST IF is markedly reduced. FIG. 15D shows simultaneous binding affinity between the two formats, where TS IF improves over TS IF by more than one order of magnitude.

FIGS. 16A-16C evaluates how variance in binding between ST IF and TS IF impact tumor progression and survival in 4T1 orthotopic mouse model, where groups were treated with either control antibody, a cocktail of anti-S15 and anti-CD3 IgG, ST IF, or TS IF. FIG. 16A shows tumor size over the study period. Arrows indicate treatment time points. FIG. 16B shows the Kaplan Meier survival curves of all groups. FIG. 16C shows tumor weight in a subset of mice from each group, sacrificed 9 days after final treatment.

FIGS. 17A-17H evaluate modulation of the tumor microenvironment 7 days after the last treatment. The tumor was harvested, homogenized, and evaluated by flow cytometry for various immune cells. FIGS. 17A-17C show percentages of helper T-cells, regulatory T-cells, and killer T-cells, respectively. FIGS. 17D-17F show the presence of myeloid-derived suppressor cells, type two macrophages, and the ratio of type one to two macrophages present, respectively. FIG. 17G shows the presence of activated T-cells within the tumor, and FIG. 17H shows the presence of dendritic cells within the tumor.

FIGS. 18A-18H evaluate modulation of the tumor microenvironment 14 days after the last treatment. The tumor was harvested, homogenized, and evaluated by flow cytometry for various immune cells. FIGS. 18A-18C show percentages of helper T-cells, regulatory T-cells, and killer T-cells, respectively. FIGS. 18D-18F show the presence of myeloid-derived suppressor cells, type two macrophages, and the ratio of type one to two macrophages present, respectively. FIG. 18G shows the presence of activated T-cells within the tumor, and FIG. 18H shows the presence of dendritic cells within the tumor.

FIGS. 19A-19H evaluate modulation of the tumor microenvironment at the study endpoint 20 days after the last treatment. The tumor was harvested, homogenized, and evaluated by flow cytometry for various immune cells. FIGS. 20A-20C show the percentages of helper T-cells, regulatory T-cells, and killer T-cells, respectively. FIGS. 20D-20F show the presence of myeloid-derived suppressor cells, type two macrophages, cancer stem cells, respectively. FIG. 20G shows the presence of activated T-cells within the tumor, and FIG. 20H shows the presence of dendritic cells within the tumor.

FIGS. 20A-20G evaluate modulation of systemic anti-tumor response 14 days after the last treatment. Lymph nodes were harvested, homogenized, and evaluated by flow cytometry for various immune cells. FIGS. 20A-20C show percentages of helper T-cells, regulatory T-cells, and killer T-cells, respectively. FIGS. 20D-20F show the presence of myeloid-derived suppressor cells, type two macrophages, and activated T-cells, respectively. FIG. 20G shows the presence of dendritic cells.

FIGS. 21A-21G evaluate modulation of systemic anti-tumor response at the study endpoint 20 days after the last treatment. Lymph nodes were harvested, homogenized, and evaluated by flow cytometry for various immune cells. FIGS. 21A-21C show percentages of helper T-cells, regulatory T-cells, and killer T-cells, respectively. FIGS. 21D-21F show the presence of myeloid-derived suppressor cells, type two macrophages, and activated T-cells, respectively. FIG. 21G shows the presence of dendritic cells.

FIGS. 22A-22D show modulation of TGFβ1 and TGFβ2 levels within the tumor, as indicated. Tumor tissue was harvested and homogenized with RIPI buffer. Tumor lysates were collected, and TGFβ levels evaluated by Luminex assay at 7 days post final treatment (FIGS. 22A and 22B) and at the study end-point, 20 days post final treatment (FIGS. 22C and 22D).

FIGS. 23A-23F show modulation of cytokine levels within the tumor. Tumor tissue was harvested and homogenized with RIPI buffer. Tumor lysates were collected, and cytokine levels evaluated by Luminex assay. FIGS. 23A-23C show levels of IL-6, IL-10, and IFNγ, respectively, at 7 days post final treatment. FIGS. 23D-23F show levels of IL-6, IL-10, and IFNγ, respectively, at the study end-point 20 days post final treatment.

### DETAILED DESCRIPTION

Disclosed herein are methods, compositions, systems, and kits for treating or delaying the progression of cancer. The ideal tumor microenvironment (TME) immunomodulatory intervention should target an antigen that is highly expressed on tumor, with minimal expression on healthy tissue, thereby enabling highly selective accumulation in the target tumor and inhibit tumor induced immunosuppression of antigen presenting cells and T cell function within TME. Described herein is a bispecific antibody that targets both Siglec15 and TGF-β and methods of using thereof. Surprisingly, the bispecific antibody was highly effective at suppressing tumor growth, with resulted in a clear survival benefit. Importantly, the effectiveness of the bispecific antibody was far greater than targeting S15 alone, targeting TGF-β alone, and even a cocktail of both monospecific antibodies, see Example 4. Consistent with greater anti-tumor efficacy, the bispecific antibody treatment normalized the dysregulated anti-tumor response and further rebuilt the immunosupportive TME. Thus, Siglec15 is a target for developing bispecific molecules for modulation of the tumor immune microenvironment and for more effective cancer immunotherapy.

Section headings as used in this section and the entire disclosure herein are merely for organizational purposes and are not intended to be limiting.

### 1. DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Antibody” and “antibodies” as used herein refers to monoclonal antibodies, monospecific antibodies (e.g., which can either be monoclonal, or may also be produced by other means than producing them from a common germ cell), multi-specific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25(11):1290-1297 (2007) and PCT International Application WO 2001/058956, the contents of each of which are herein incorporated by reference), or domain antibodies (dAbs) (e.g., such as described in Holt et al., Trends in Biotechnology 21:484-490 (2014)), and including single domain antibodies sdAbs that are naturally occurring, e.g., as in cartilaginous fishes and camelid, or which are synthetic, e.g., nanobodies, VHH, or other domain structure), and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2), or subclass. For simplicity sake, an antibody against an analyte is frequently referred to herein as being either an “anti-analyte antibody” or merely an “analyte antibody”.

“Antibody fragment” as used herein refers to a portion of an intact antibody that retain the ability to specifically bind to an antigen (see, generally, Holliger et al., Nat. Biotech., 23(9): 1126-1129 (2005)) (e.g., comprises the antigen-binding site or variable region). Any antigen-binding fragment of the antibody described herein is within the scope of the present disclosure. The antibody may not include the constant heavy chain domains (e.g., CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)₂ fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

Typically, an immunoglobulin or antibody is a protein that comprises at least one complementarity determining region (CDR). The CDRs form the “hypervariable region” of an antibody, which is responsible for antigen binding (discussed further below). A whole antibody typically consists of four polypeptides: two identical copies of a heavy (H) chain polypeptide and two identical copies of a light (L) chain polypeptide. Each of the heavy chains contains one N-terminal variable (V_{H}) region and three C-terminal constant (C_{H1}, C_{H2}, and C_{H3}) regions, and each light chain contains one N-terminal variable (V_{L}) region and one C-terminal constant (C_{L}) region. The light chains of antibodies can be assigned to one of two distinct types, either kappa (κ) or lambda (λ), based upon the amino acid sequences of their constant domains. In a typical antibody, each light chain is linked to a heavy chain by disulfide bonds, and the two heavy chains are linked to each other by disulfide bonds. The light chain variable region is aligned with the variable region of the heavy chain, and the light chain constant region is aligned with the first constant region of the heavy chain. The remaining constant regions of the heavy chains are aligned with each other.

The variable regions of each pair of light and heavy chains form the antigen binding site of an antibody. The V_{H} and V_{L} regions have the same general structure, with each region comprising four framework (FW or FR) regions. The term “framework region,” as used herein, refers to the relatively conserved amino acid sequences within the variable region which are located between the CDRs. There are four framework regions in each variable domain, which are designated FR1, FR2, FR3, and FR4. The framework regions form the β sheets that provide the structural framework of the variable region (see, e.g., C. A. Janeway et al. (eds.), Immunobiology, 5th Ed., Garland Publishing, New York, N.Y. (2001)).

“CDR” is used herein to refer to the “complementarity determining region” within an antibody variable sequence. There are three CDRs in each of the variable regions of the heavy chain and the light chain. Proceeding from the N-terminus of a heavy or light chain, these regions are denoted “CDR1,” “CDR2,” and “CDR3,” for each of the variable regions. The term “CDR set” as used herein refers to a group of three CDRs that occur in a single variable region that binds the antigen. An antigen-binding site, therefore, may include six CDRs, comprising the CDR set from each of a heavy and a light chain variable region. A polypeptide comprising a single CDR, (e.g., a CDR1, CDR2, or CDR3) may be referred to as a “molecular recognition unit.” Crystallographic analyses of antigen-antibody complexes have demonstrated that the amino acid residues of CDRs form extensive contact with bound antigen, wherein the most extensive antigen contact is with the heavy chain CDR3. Thus, the molecular recognition units may be primarily responsible for the specificity of an antigen-binding site. In general, the CDR residues are directly and most substantially involved in influencing antigen binding.

The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as “Kabat CDRs”. Chothia and coworkers (Chothia and Lesk, J. Mol. Biol., 196: 901-917 (1987); and Chothia et al., Nature, 342: 877-883 (1989)) found that certain sub-portions within Kabat CDRs adopt nearly identical peptide backbone conformations, despite having great diversity at the level of amino acid sequence. These sub-portions were designated as “L1,” “L2,” and “L3,” or “H1,” “H2,” and “H3,” where the “L” and the “H” designate the light chain and the heavy chain regions, respectively. These regions may be referred to as “Chothia CDRs,” which have boundaries that overlap with Kabat CDRs. Other boundaries defining CDRs overlapping with the Kabat CDRs have been described by Padlan, FASEB J., 9: 133-139 (1995), and MacCallum, J. Mol. Biol., 262(5): 732-745 (1996). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain aspects use Kabat- or Chothia-defined CDRs.

As used herein, when an antibody or other entity (e.g., antigen binding domain) “specifically recognizes” or “specifically binds” an antigen or epitope, it preferentially recognizes the antigen in a complex mixture of proteins and/or macromolecules, and binds the antigen or epitope with affinity which is substantially higher than to other entities not displaying the antigen or epitope. In this regard, “affinity which is substantially higher” means affinity that is high enough to enable detection of an antigen or epitope which is distinguished from entities using a desired assay or measurement apparatus. Typically, it means binding affinity having a binding constant (Kₐ) of at least 10⁷ M⁻¹ (e.g., >10⁷ M⁻¹, >10⁸ M⁻¹, >10⁹ M⁻¹, >10¹⁰ M⁻¹, >10¹¹ M⁻¹, >10¹² M⁻¹, >10¹³ M⁻¹, etc.). In certain such embodiments, an antibody is capable of binding different antigens so long as the different antigens comprise that particular epitope. In certain instances, for example, homologous proteins from different species may comprise the same epitope.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (KD). Affinity can be measured by common methods known in the art, including those described herein. Specific illustrative and exemplary embodiments for measuring binding affinity are described in the following.

The term “monoclonal antibody,” as used herein, refers to an antibody produced by a single clone of B lymphocytes that is directed against a single epitope on an antigen. Monoclonal antibodies typically are produced using hybridoma technology, as first described in Köhler and Milstein, Eur. J. Immunol., 5: 511-519 (1976). Monoclonal antibodies may also be produced using recombinant DNA methods (see, e.g., U.S. Pat. No. 4,816,567), isolated from phage display antibody libraries (see, e.g., Clackson et al. Nature, 352: 624-628 (1991)); and Marks et al., J Mol. Biol., 222: 581-597 (1991)), or produced from transgenic mice carrying a fully human immunoglobulin system (see, e.g., Lonberg, Nat. Biotechnol., 23(9): 1117-25 (2005), and Lonberg, Handb. Exp. Pharmacol., 181: 69-97 (2008)). In contrast, “polyclonal” antibodies are antibodies that are secreted by different B cell lineages within an animal. Polyclonal antibodies are a collection of immunoglobulin molecules that recognize multiple epitopes on the same antigen.

The term “monospecific” antibody as used herein denotes an antibody that has one or more binding sites each of which bind to the same epitope of the same antigen.

The term “bispecific” antibody as used herein denotes an antibody that has at least two binding sites each of which bind to different epitopes of the same antigen or a different antigen.

The term “multispecific” antibody as used herein denotes an antibody that has binding specificities for at least two different sites.

The term “valent” as used within the current application denotes the presence of a specified number of binding sites in an antibody molecule. As such, the terms “bivalent,” “tetravalent,” and “hexavalent” denote the presence of two binding sites, four binding sites, and six binding sites, respectively, in an antibody molecule. The bispecific antibodies according to the invention are at least “bivalent” and may be “trivalent” or “multivalent” (e.g., “tetravalent” or “hexavalent”). That is, the antibodies may be bispecific even in cases where there are more than two binding sites (e.g., that the antibody is trivalent or multivalent).

The terms “immunogen” and “antigen” are used interchangeably herein and refer to any molecule, compound, or substance that induces an immune response in an animal (e.g., a mammal). An “immune response” can entail, for example, antibody production and/or the activation of immune effector cells. An antigen in the context of the disclosure can comprise any subunit, fragment, or epitope of any proteinaceous or non-proteinaceous (e.g., carbohydrate or lipid) molecule that provokes an immune response in a mammal. By “epitope” is meant a sequence of an antigen that is recognized by an antibody or an antigen receptor. Epitopes also are referred to in the art as “antigenic determinants.” In certain embodiments, an epitope is a region of an antigen that is specifically bound by an antibody. In certain embodiments, an epitope may include chemically active surface groupings of molecules such as amino acids, sugar side chains, phosphoryl, or sulfonyl groups. In certain embodiments, an epitope may have specific three-dimensional structural characteristics (e.g., a “conformational” epitope) and/or specific charge characteristics. The antigen can be a protein or peptide of viral, bacterial, parasitic, fungal, protozoan, prion, cellular, or extracellular origin, which provokes an immune response in a mammal, preferably leading to protective immunity.

A “chemotherapeutic” or “chemotherapeutic agent” as used herein, refers to a chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinase inhibitors. Chemotherapeutic agents include compounds used in “targeted therapy” and conventional chemotherapy. Examples of chemotherapeutic agents include, but are not limited to: cyclophosphamide, methotrexate, 5-fluorouracil, doxorubicin, docetaxel, daunorubicin, bleomycin, vinblastine, dacarbazine, cisplatin, carboplatin, paclitaxel, raloxifene hydrochloride, tamoxifen citrate, abemacicilib, everolimus, alpelisib, anastrozole, pamidronate, anastrozole, exemestane, capecitabine, epirubicin hydrochloride, eribulin mesylate, erlotinib, toremifene, fulvestrant, letrozole, gemcitabine, goserelin, ixabepilone, emtansine, lapatinib, olaparib, megestrol, neratinib, palbociclib, ribociclib, talazoparib, thiotepa, toremifene, methotrexate, trastuzumab, temozolomide, rapamycin, and tucatinib.

A “peptide” or “polypeptide” is a linked sequence of two or more amino acids linked by peptide bonds. Peptides and polypeptides include proteins such as binding proteins, receptors, and antibodies. The terms “polypeptide” and “protein” are used interchangeably herein.

As used herein, the terms “providing,” “administering,” and “introducing” are used interchangeably herein and refer to the placement into a subject by a method or route which results in at least partial localization to a desired site.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment, the mammal is a human.

As used herein, “treat,” “treating,” and the like means a slowing, stopping, or reversing of progression of a disease or disorder when provided a bispecific binding molecule or composition described herein to an appropriate subject. The term also includes a reversing of the progression of such a disease or disorder to a point of eliminating or greatly reducing the disease. As such, “treating” means an application or administration of the bispecific binding molecule or compositions described herein to a subject, where the subject has a disease or a symptom of a disease, where the purpose is to cure, heal, alleviate, relieve, alter, remedy, ameliorate, improve, or affect the disease or symptoms of the disease.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. METHODS

The present disclosure provides methods for treating cancer or delaying the progression of cancer in a subject in need thereof. The methods comprise administering to a subject in need thereof an effective amount of a bispecific binding molecule configured to specifically bind a cancer cell membrane-associated target and one of an anti-inflammatory cytokine or chemokine, a tumorigenic cytokine or chemokine, or a T cell activator.

A cancer cell membrane-associated target includes any peptide, polypeptide, protein, or other molecule expressed on the surface of a cancer cell or overexpressed on the surface of a cancer cell in comparison to non-diseased cells. Generally, all membrane proteins that are overexpressed on cancer or cancer-associated cells are potentially suitable targets of the bispecific binding molecule. Cancer cell membrane-associated targets include, but are not limited to Carbonic anhydrase-9 (CAIX), Carcinoembryonic antigen (CEA), CD13, CD44, CXCR4, epidermal growth factor receptor (EGFR), Extracellular matrix metalloproteinase inducer (EMMPRIN), Endoglin, Epithelial cell adhesion molecule (EpCAM), EphA2, epidermal growth factor receptor-2 (ErbB2), Fibroblastic activation protein-α (FAP-α), Folate receptor, Glucose-regulated protein-78 (GFP78), Insulin-like growth factor-1 receptor (IGF-1R), Matriptase, Mesothelin, hepatocyte growth factor receptor (cMET/HGFR), membrane type-1 matrix metalloproteinase (MT1-MMP), membrane type-6 matrix metalloproteinase (MT6-MMP), Mucin-1 (Muc-1), Prostate stem cell antigen (PSCA), Prostate-specific membrane antigen (PSMA), Siglec15 (S15), Urokinase-type plasminogen activator receptor (uPAR). In some embodiments, the is Siglec15 (S15).

In some embodiments, the anti-inflammatory or tumorigenic chemokine or cytokine is selected from the group consisting of: TGF-β; IL-1 receptor agonist; IL-4; IL-10; IL-11; IL-13; IL-19; IL-20; IL-22; IL-23; IL-24; IL-26; IL-27; IL-28; IL-29; IL-33; IL-35; IL-37; IFN-α; IFN-γ; and CXCL12. In select embodiments, the anti-inflammatory or tumorigenic chemokine or cytokine is TGF-β.

An “T cell activator,” as used herein, refers to a peptide, polypeptide, protein, or other molecule expressed on the surface of a T lymphocyte, particularly a cytotoxic T lymphocyte, which is capable of inducing T cell activation upon interaction with an antigen binding molecule. Specifically, interaction of a binding molecule with an T cell activator may induce T cell activation by triggering the signaling cascade of the T cell receptor complex. “T cell activation,” as used herein, refers to one or more cellular response of a T lymphocyte, particularly a cytotoxic T lymphocyte, selected from: proliferation, differentiation, cytokine secretion, cytotoxic effector molecule release, cytotoxic activity, and expression of activation markers. The T cell activating bispecific antigen binding molecules of the invention are capable of inducing T cell activation. Suitable assays to measure T cell activation are known in the art described herein. In some embodiments, the T cell activator is selected from the group consisting of: CD2, CD3, and CD28. In some embodiments, T cell activators include those molecules that can redirect T cells to the cancer cells without the classical activation pathways, such as CD4 and CD8.

In select embodiments, the T cell activator is CD3. “CD3,” as used herein, refers to a protein complex composed of four distinct chains: gamma, delta, and two epsilon chains. These chains associate with the T cell receptor (TCR) and the zeta-chain to generate an activation signal in T lymphocytes. The term “CD3” encompasses “full-length,” unprocessed CD3 as well as any form of CD3 that results from processing in the cell. In some embodiments, the binding molecule specifically binds an epsilon subunit of CD3. In other embodiments, the binding molecule specifically binds the alpha or beta subunit of CD3.

The methods encompass administration of two or more (e.g., 2, 3, 4, 5, 6, or more) bispecific binding molecules as described herein. For example, the methods may include administration of a first bispecific binding molecule and a second bispecific binding molecule. In some embodiments, the first bispecific binding molecule specifically binds Siglec15 (S15) and an anti-inflammatory cytokine or chemokine, a tumorigenic cytokine or chemokine. In some embodiments, the first bispecific binding molecule specifically binds Siglec15 (S15) and TGF-β. In some embodiments, the second bispecific binding molecule specifically binds Siglec15 (S15) and a T cell activator. In some embodiments, the second bispecific binding molecule specifically binds Siglec15 (S15) and CD3.

In some embodiments, the subject is a human. In some embodiments, the subject has cancer or has had cancer.

The methods disclosed herein are suitable for any cancer type or subtype. In some embodiments, the cancer may be a carcinoma, sarcoma, lymphoma, leukemia, melanoma, mesothelioma, multiple myeloma, or seminoma. The cancer may be a cancer of the bladder, blood, bone, brain, breast, cervix, colon/rectum, endometrium, head and neck, kidney, liver, lung, muscle tissue, ovary, pancreas, prostate, skin, spleen, stomach, testicle, thyroid, or uterus. In some embodiments, the cancer is breast cancer, liver cancer, lung cancer, or pancreatic cancer.

In select embodiments, the cancer is breast cancer. Breast cancer is often classified into three major categories based on the combination of receptors expression: hormone receptor (estrogen or progesterone) positive/HER2 negative (HR+/HER2−), HER2 positive/hormone receptor positive or negative (HER2+/HR− or HR+), and triple-negative (HR−/HER2−). In exemplary embodiments, the cancer is triple negative breast cancer.

In select embodiments, the cancer is pancreatic cancer.

In some embodiments, the cancer comprises a solid tumor. In some embodiments, the methods result in decreased tumor growth. In some embodiments, the methods result in tumor regression. In some embodiments, the methods result in decreased numbers of tumors. In some embodiments, the methods prevent tumor recurrence.

In some embodiments, the cancer is invasive and/or metastatic cancer (e.g., stage II cancer, stage III cancer or stage IV cancer). In some embodiments, the cancer is an early stage cancer (e.g., stage 0 cancer, stage I cancer), and/or is not invasive and/or metastatic cancer. In some embodiments, the methods result in decreased metastasis. In some embodiments, the methods prevent metastasis.

In some embodiments, the methods result in increases in overall subject survival.

In some embodiments, the methods result in T cell activation. Thus, also disclosed herein are methods of activating T cells comprising administering to a subject in need thereof an effective amount of a bispecific binding molecule configured to specifically bind a cancer cell membrane-associated target and one of an anti-inflammatory cytokine or chemokine, a tumorigenic cytokine or chemokine, or a T cell activator.

In some embodiments, the methods disclosed herein utilizing the bispecific binding molecule had increased effectiveness as compared to antibodies against the cancer cell membrane-associated target and one of an anti-inflammatory cytokine or chemokine, a tumorigenic cytokine or chemokine, or a T cell activator alone or in combination. Thus, the disclosed methods may provide increased effectiveness in treating cancer or delaying cancer progression, as measured by the results described herein, as compared to methods comprising administration of an antibody against the cancer cell membrane-associated target, as compared to methods comprising administration of an antibody against the anti-inflammatory cytokine or chemokine, the tumorigenic cytokine or chemokine, or the T cell activator, or as compared to methods comprising administration of an antibody against the cancer cell membrane-associated target and a separate antibody against the anti-inflammatory cytokine or chemokine, the tumorigenic cytokine or chemokine, or the T cell activator.

An “effective amount” is an amount that is delivered to a subject, either in a single dose or as part of a series, which is effective for inducing a response in the subject. This amount varies depending upon the health and physical condition of the subject to be treated, the relative stage of the cancer, and other relevant factors. It is expected that the amount will fall in a relatively broad range that can be determined by one of skill in the art through routine trials.

The bispecific binding molecule described herein can be administered in a wide variety of therapeutic dosage forms. The route and regimen of administration will vary depending upon the population and is to be determined by the skilled practitioner. For example, the formulations, compositions, or medicaments disclosed herein may be administered parentally, e.g., intravenous (either by bolus or infusion methods), intraperitoneal, subcutaneous, topical with or without occlusion, or intramuscular form.

In any of the uses or methods described herein, administration may be by various routes known to those skilled in the art, including without limitation oral, inhalation, intravenous, intramuscular, topical, subcutaneous, systemic, and/or intraperitoneal administration to a subject in need thereof. In some embodiments, administration includes parenteral administration (including, but not limited to, subcutaneous, intramuscular, intravenous, intraperitoneal, intracardiac and intraarticular injections). In some embodiments, the administration is systemic. In some embodiments, the administration is intratumoral or adjacent to the site of cancer.

The specific dose level may depend upon a variety of factors including the activity of the polynucleotides (e.g., mRNA), polypeptides, compositions, formulations, vaccine, or medicament, the age, body weight, general health, and diet of the subject, time of administration, and route of administration. For prophylaxis purposes, the amount in each dose is an amount which induces an immunoprotective response without significant adverse side effects.

A wide range of second therapies may be used in conjunction with the bispecific binding molecule, formulations, compositions, medicaments, and methods of the present disclosure. The second therapy may be administration of a therapeutic agent or may be a second therapy not connected to administration of another agent. Such second therapies include, but are not limited to, surgery, radiotherapy, or a chemotherapeutic or anti-cancer agent. In select embodiments, the method further comprises administration of radiation therapy.

The second therapy may be administered at the same time as the initial therapy. The second therapy may be administered in the same composition or in a separate composition administered at substantially the same time as the initial therapy. In some embodiments, the second therapy may precede or follow administration of the disclosed bispecific binding molecule, formulations, compositions, medicaments, and methods by time intervals ranging from hours to months.

In some embodiments, a therapeutically effective amount of a bispecific binding molecule, disclosed herein, or compositions thereof, is administered alone or in combination with a therapeutically effective amount of at least one additional therapeutic agent. In some embodiments, effective combination therapy is achieved with a single composition or pharmacological formulation that includes both agents, or with two distinct compositions or formulations, administered at the same time or separated by a time interval, wherein one composition includes a compound of this invention, and the other includes the at least one additional therapeutic agent. In select embodiments, the at least one additional therapeutic agent comprises a chemotherapeutic or anti-cancer agent.

In select embodiments, the methods further comprise full or partial resection of a tumor or cancerous tissue or organ. In some embodiments, bispecific binding molecule is administered prior to the resection. In some embodiments, the bispecific binding molecule is administered prior to and following the resection.

A typical dose of a therapeutically effective amount of a bispecific binding molecule of the present disclosure (e.g., an anti-S15/anti-TGF-β bispecific antibody, an anti-S15/anti-CD3 bispecific antibody) can range from, for example, about 0.0001 mg/dose to about 100 mg/dose for each treatment.

In addition to therapeutic uses, a bispecific binding molecule (e.g., a bispecific antibody or antigen-binding fragment, as described herein) can be used in diagnostic or research applications. Research applications include, for example, methods that utilize the bispecific binding molecule and a label to detect the corresponding antigen(s) in a sample, e.g., in a human body fluid or in a cell or tissue extract. The bispecific binding molecule thereof may be employed in any suitable assay for measuring a corresponding antigen(s) in a sample for diagnostic and/or research purposes. Such assays include, but are not limited to, sandwich immunoassays, enzyme immunoassays (EIA), enzyme-linked immunosorbent assays (ELISA), lateral flow assays, competitive inhibition immunoassays (e.g., forward and reverse), competitive binding assays, Forster resonance energy transfer (FRET), one-step antibody detection assays, single molecule detection assays, radioimmunoassays (RIA), and FACS. Such methods are disclosed in, for example, U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792; and Adamczyk et al., Anal. Chim. Acta, 579(1): 61-67 (2006).

### 3. BISPECIFIC ANTIBODIES

In some embodiments, the bispecific binding molecule is a bispecific antibody, or fragment thereof, having one or more antigen binding sites directed against S15 and one or more antigen binding sites directed against one of the anti-inflammatory cytokine or chemokine, the tumorigenic cytokine or chemokine, or the T cell activator.

A bispecific antibody of the present disclosure may be a whole antibody, or an antigen-binding fragment of a whole antibody. As defined herein, antigen-binding antibody fragments encompassed by the present disclosure include, but are not limited to, F(ab′)2, Fab′, Fab, Fv, scFv, dsFv, dAb, and single chain binding polypeptides. Antibody fragments and their therapeutic utility are further described in, e.g., Nelson, A. L., MAbs. 2010 January-February; 2(1): 77-83; Joosten et al., Microbial Cell Factories volume 2, Article number: 1 (2003); and Bates A, Power CA., Antibodies (Basel). 2019; 8(2):28; doi:10.3390/antib8020028). In some embodiments, the anti-Siglec15 and/or the anti-inflammatory or tumorigenic cytokine or chemokine (e.g., anti-TGF-β, anti-CD3) antigen-binding fragments are a single-chain variable fragments (scFv), which are engineered antibodies generated by the fusion of the heavy (VH) and light chains (VL) of immunoglobulins through a short polypeptide linker. Single chain variable domain (Fv) fragments (scFv) are used in the art in a variety of clinical and therapeutic applications, primarily due to their improved pharmacokinetic properties as compared to the parent monoclonal antibodies and the relative ease of producing them in large quantities at low cost (Monnier et al., Antibodies 2013, 2(2), 193-208; doi.org/10.3390/antib2020193; Safdari et al., Mol Med. 2016; 22: 258-270; and Lu, R., Hwang, Y., Liu, I. et al. Development of therapeutic antibodies for the treatment of diseases. J Biomed Sci 27, 1 (2020). https://doi.org/10.1186/s12929-019-0592-z).

The bispecific antibodies of the present disclosure may be a diabody. Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

In other embodiments, a bispecific antibody of the present disclosure is a whole antibody. As defined herein, a whole antibody comprises two identical copies of a heavy (H) chain polypeptide and two identical copies of a light (L) chain polypeptide. Each of the heavy chains contains one N-terminal variable (VH) region and three C-terminal constant (CH1, CH2, and CH3) regions, and each light chain contains one N-terminal variable (VL) region and one C-terminal constant (CL). The heavy chain C-terminal constant region contains the fragment crystallizable (Fc) domain, which determines antibody class and is responsible for humoral and cellular effector functions. Antibodies are divided into five major classes (or “isotypes”), IgG, IgM, IgA, IgD and IgE, which differ in their function in the immune system. IgGs are the most abundant immunoglobulins in the blood, representing 60% of total serum antibodies in humans. IgG antibodies may be subclassified as IgG1, IgG2, IgG3, and IgG4, named in order of their abundance in serum (IgG1 being the most abundant) (Vidarsson et al., Frontiers in Immunology. 5: 520 (2014)). A whole bispecific antibody described herein (e.g., an anti-Siglec15/anti-TGF-β bispecific antibody, an anti-Siglec15/anti-CD3 bispecific antibody) may be of any suitable class and/or subclass. In some embodiments, the antibody is of class IgG (e.g., IgG1, IgG2, IgG3, or IgG4). For example, the antibody may be an IgG1 antibody.

As described above, the bispecific antibodies of the present disclosure can be a monoclonal antibody, a human antibody, a humanized antibody, and/or a chimeric antibody. In some embodiments, the antibody is a fragment selected from the group consisting of Fab, Fab-C, Fab′-SH, Fv, scFv, and (Fab′)2 fragments. In some embodiments, the bispecific antibodies of the present disclosure comprise two or more single-domain antibodies that form a bivalent antibody, a trivalent antibody, or a tetravalent antibody that recognizes different epitopes on the same or different antigens.

In some embodiments, the bispecific antibodies of the present disclosure are chimeric antibodies. Certain chimeric antibodies are described, for example, in U.S. Pat. No. 4,816,567; and Morrison et al., Proc. Natl. Acad. Sci. USA. 81:6851-6855 (1984). In one example, a chimeric antibody comprises a non-human variable region (e.g., a variable domain derived from a mouse, rat, hamster, rabbit, or non-human primate, such as a monkey) and a human constant domain. In some embodiments chimeric antibody comprise binding domains derived from non-human organisms in an otherwise humanized antibody, or alternatively, human binding domains in an otherwise non-human antibody. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, for example, CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, for example, in Almagro and Fransson, Front. Biosci. 13:1619-1633 (2008), and are further described, for example, in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

In accordance with the above embodiments, the bispecific antibodies of the present disclosure can be made into bivalent, trivalent, or tetravalent formats, e.g., having two or more binding sites for one or both of the antigens. For example, a bispecific antibody of the present disclosure can be a bivalent, bispecific antibody with heteromeric heavy chains (e.g., Triomab, knobs-into-holes (KIH), Duobody, etc). A bispecific antibody of the present disclosure can be a tetravalent antibody comprised of IgGs with other binding domains fused to either the N- or C-termini of either the heavy or light chains (e.g., dual variable domain [DVD], IgG-scFv fusion, Mabtyrin (IgG with non-antibody binding scaffold “centyrin” fused to C-terminal end of heavy chains). A bispecific antibody of the present disclosure can be comprised of IgGs to which additional antigen combining sites have been added within the structure (e.g., two-in-one antibodies, MAT “Modular Antibody Technology” platform from F-Star). A bispecific antibody of the present disclosure can be an engineered antibody fragment linked by short peptide linkers which can be made into bivalent, trivalent, or tetravalent formats addressing two to three targets (e.g., bispecific T-cell engager (BiTE), Nanobody platform, dual-affinity re-targeting (DART) antibodies, “tandem antibody” structures (TandAbs)). And a bispecific antibody of the present disclosure can be comprised of chemically coupled IgGs.

In general, there are more than 100 different bispecific antibody formats that have been produced due to the modular architecture of antibodies. These formats vary in many ways, including based on their molecular weight, the number of antigen-binding sites, the spatial relationship between different binding sites, the valency for each antigen, the ability to support secondary immune functions, and the pharmacokinetic half-life. Recombinant bispecific antibodies can be divided into two classes: bispecific formats with Fc regions, and bispecific formats without Fc regions. Bispecific antibodies with an Fc region retain Fc-mediated effector functions. These formats include, but are not limited to, “knob into hole” IgG, crossMab, ortho-Fab IgG, DVD-Ig, two in one IgG, IgG-scFv and scFv2-Fc. The Fc regions used in the bispecific antibodies disclosed herein may be from any IgG subtype (e.g., IgG1, IgG2, IgG3, IgG4) but also contain mutations (e.g., substitutions, additions, or deletions of one or more amino acids). In some embodiments, the Fc region comprises mutations, for example to improve binding, improve stability, improve production and/or purification, or any combination thereof

Bispecific antibodies with no Fc lack Fc-mediated effector functions. However, the smaller size of such antibodies offers a better tumor tissue penetration over IgG-like formats. In this format, the variable domains of each parental monoclonal antibody and the linkers are cloned and linked to form a single-chain bispecific antibody. These bispecific antibodies represent many formats, including tandem scFvs, diabody format, single-chain diabodies, tandem diabodies (TandAbs), dual-affinity retargeting molecules (DARTs), dock-and-lock (DNL), and nanobodies.

A number of strategies have been developed to generate bispecific antibodies. Hybrid hybridoma (also referred to as quadroma) was the earliest technology used to produce bispecific antibodies. It is based on the somatic fusion of two different hybridoma cell lines expressing murine IgGs of desired specificities. However, the real percentage of functional bispecific antibody by a quadroma cell line is unpredictable and a laborious process is required to isolate the bispecific antibody from the side products. By using molecular cloning technology, bispecific IgG antibodies can be assembled from two different heavy and light chains expressed in the same producer cell. The production of bispecific antibodies requires at least two plasmids for heterodimerized heavy chains and one plasmid for a common light chain or two light-chain plasmids if two different light chains are used. Notably, expressing HC and LC on separate plasmids can be advantageous because the manipulation of the plasmid ratio is an easy and efficient approach to optimize protein assembly for desired products. Subsequently, a laborious and time-consuming process is typically needed to select the most desirable clonal cell lines from a heterogeneous stable transfectant pool for large-scale antibody production. Generally, methods of making bispecific antibodies that can be divided into three groups: chemical recombination, cell fusion, and genetic manipulation (Reichert, J. M, and Dhimolea, E., Drug Discov Today, 2012 September; 17(17-18):954-63). Recent reviews on the different strategies in constructing bispecific antibodies, with therapeutic application emphasis, include Wu, C., Drug News Perspect. 2009 October; 22(8):453-8; Gu, J., and Ghayur, T., Methods Enzymol. 2012; 502:25-41; and Kontermann, R., MAbs, March-April 2012; 4(2): 182-97.

In some embodiments, the bispecific antibody comprises one or more V_{H} domains each comprising an HCDR1, an HCDR2, and/or an HCDR3, and one or more V_{L} domains each comprising an LCDR1, an LCDR2, and/or an LCDR3 from an anti-Siglec15 antibody. For example, anti-S15 antibodies include, but are not limited to those, disclosed or referenced in: He, F., et al., J Clin Transl Res. 2021 Dec. 28; 7(6): 739-749, Wu, J., Molecular Pharmacology Jun. 28, 2022, MOLPHARM-AR-2021-000470; DOI: doi.org/10.1124/molpharm.121.000470, Zhen, G., Bone Res 9, 47 (2021). doi.org/10.1038/s41413-021-00161-1, Tsukazaki, H., et al., Bone, Volume 152, 2021, 116095, doi.org/10.1016/j.bone.2021.116095, and Cao, H., (2021) Br. J. Haematol., 193: 946-950. doi.org/10.1111/bjh.17496.

In some embodiments, the bispecific antibody comprises one or more V_{H} domains each comprising an HCDR1, an HCDR2, and/or an HCDR3, and one or more V_{L} domains each comprising an LCDR1, an LCDR2, and/or an LCDR3 from an antibody directed to an anti-inflammatory cytokine or chemokine, a tumorigenic cytokine or chemokine, or a T cell activator. For example, anti-CD3 antibodies include, but are not limited to, those disclosed or referenced in: Kuhn and Weiner, Immunotherapy 2016 8:8, 889-906, Chatenoud and Waldmann Rev Diabet Stud. 2012 Winter; 9(4): 372-381, Herold, K. C. et al. N. Engl. J. Med. 346, 1692-1698 (2002), Smith, J. A. et al. J. Exp. Med. 185, 1413-1422 (1997), and Bisikirska, B. et al. J. Clin. Invest. 115, 2904-2913 (2005). For example, anti-TGF-β antibodies include, but are not limited to, those disclosed or referenced in: Arteaga, C L, et al., J Clin Invest. 1993 December; 92(6): 2569-2576, Lonning, S. et al., Current Pharmaceutical Biotechnology, Volume 12, Number 12, 2011, pp. 2176-2189(14), Gagliardini and Benigni Cytokine & Growth Factor Reviews, Volume 17, Issues 1-2, 2006, Pages 89-96, doi.org/10.1016/j.cytogfr.2005.09.005 and Biswas, S., et al., J Clin Invest. 2007; 117(5):1305-131, doi.org/10.1172/JCI30740.

Table 1 provides references disclosed V_{H} domains (e.g., HCDR1, HCDR2, HCDR3) and V_{L}domains (e.g., LCDR1, LCDR2, LCDR3) directed to Siglec15, anti-inflammatory cytokines or chemokines, tumorigenic cytokines or chemokines, and T cell activators suitable for use in the bispecific antibodies disclosed herein.

TABLE 1

| Target | Reference(s) |
|---|---|
| Siglec-15 | Wu, J., Molecular Pharmacology Jun. 28, 2022, MOLPHARM-AR-2021- |
| | 000470; DOI: doi.org/10.1124/molpharm.121.000470 |
| | Zhen, G., Bone Res 9, 47 (2021). doi.org/10.1038/s41413-021-00161-1, |
| | Tsukazaki, H., et al., Bone, Volume 152, 2021, 116095, |
| | doi.org/10.1016/j.bone.2021.116095 |
| | Cao, H., (2021) Br. J. Haematol., 193: 946-950. doi.org/10.1111/bjh.17496 |
| | US20170029503A1 |
| CD3 | Kuhn and Weiner, Immunotherapy 2016 8: 8, 889-906 |
| | Chatenoud and Waldmann Rev Diabet Stud. 2012 Winter; 9(4): 372-381 |
| | Herold, K. C. et al. N. Engl. J. Med. 346, 1692-1698 (2002) |
| | Smith, J. A. et al. J. Exp. Med. 185, 1413-1422 (1997) |
| | Bisikirska, B. et al. J. Clin. Invest. 115, 2904-2913 (2005) |
| | Douglass, Jacqueline et al. “Bispecific antibodies targeting |
| | mutant RAS neoantigens.” Science immunology vol. 6, 57 (2021): eabd5515. |
| | doi: 10.1126/sciimmunol.abd5515 |
| | US20060177896 |
| | Adair, J R et al. “Humanization of the murine anti-human CD3 monoclonal |
| | antibody OKT3.” Human antibodies and hybridomas vol. 5, 1-2 (1994): 41-7. |
| | U.S. Pat. No. 4,361,549 |
| | U.S. Pat. No. 5,968,509 |
| | Thirion, S et al. “PCR cloning and sequencing of a CD3-specific monoclonal |
| | antibody.” Immunogenetics vol. 43, 3 (1996): 167-8. doi: 10.1007/BF00176681 |
| | Kipriyanov, S M et al. “Two amino acid mutations in an anti-human CD3 |
| | single chain Fv antibody fragment that affect the yield on bacterial secretion |
| | but not the affinity.” Protein engineering vol. 10, 4 (1997): 445-53. |
| | doi: 10.1093/protein/10.4.445 |
| | Cole, M S et al. “HuM291, a humanized anti-CD3 antibody, is |
| | immunosuppressive to T cells while exhibiting reduced mitogenicity in |
| | vitro.” Transplantation vol. 68, 4 (1999): 563-71. doi: 10.1097/00007890- |
| | 199908270-00020 |
| TGFβ | Arteaga, C L, et al., J Clin Invest. 1993 December; 92(6): 2569-2576 |
| | Lonning, S. et al., Current Pharmaceutical Biotechnology, Volume |
| | 12, Number 12, 2011, pp. 2176-2189(14) |
| | Gagliardini and Benigni Cytokine & Growth Factor Reviews, Volume 17, |
| | Issues 1-2, 2006, Pages 89-96, doi.org/10.1016/j.cytogfr.2005.09.005 |
| | Biswas, S., et al., J Clin Invest. 2007; 117(5): 1305-131, |
| | doi.org/10.1172/JCI30740 |
| | Grütter, Christian et al. “A cytokine-neutralizing antibody as a structural |
| | mimetic of 2 receptor interactions.” Proceedings of the National Academy of |
| | Sciences of the United States of America vol. 105, 51 (2008): 20251-6. |
| | doi: 10.1073/pnas.0807200106 |
| | U.S. Pat. No. 6,492,497 |
| | US20050049403 |
| | WO2021188749 |
| CD4 | US20060051346 |
| | Sui, Jianhua et al. “Identification of CD4 and transferrin receptor antibodies by |
| | CXCR4 antibody-guided Pathfinder selection.” European journal of |
| | biochemistry vol. 270, 22 (2003): 4497-506. doi: 10.1046/j.1432- |
| | 1033.2003.03843.x |
| | Bès, C et al. “Efficient CD4 binding and immunosuppressive properties of the |
| | 13B8.2 monoclonal antibody are displayed by its CDR-H1-derived peptide |
| | CB1.” FEBS letters vol. 508, 1 (2001): 67-74. doi: 10.1016/s0014- |
| | 5793(01)03036-8 |
| | Newman, R et al. ““Primatization” of recombinant antibodies for |
| | immunotherapy of human diseases: a macaque/human chimeric antibody |
| | against human CD4,” Bio/technology (Nature Publishing Company) vol. 10, 11 |
| | (1992): 1455-60. doi: 10.1038/nbt1192-1455 |
| | Schofield, Darren J et al. “Application of phage display to high throughput |
| | antibody generation and characterization.” Genome biology vol. 8, 11 (2007): |
| | R254. doi: 10.1186/gb-2007-8-11-r254 |
| | WO2007094983 |
| | Weissenhorn, W et al. “Combinatorial functions of two chimeric antibodies |
| | directed to human CD4 and one directed to the alpha-chain of the human |
| | interleukin-2 receptor.” Gene vol. 121, 2 (1992): 271-8. doi: 10.1016/0378- |
| | 1119(92)90131-8 |
| | Griffiths, A D et al. “Human anti-self antibodies with high specificity from |
| | phage display libraries.” The EMBO journal vol. 12, 2 (1993): 725-34. |
| | doi: 10.1002/j.1460-2075.1993.tb05706.x |
| CD8 | Gilliland, L K et al. “Rapid and reliable cloning of antibody variable regions |
| | and generation of recombinant single chain antibody fragments.” Tissue |
| | antigens vol. 47, 1 (1996): 1-20. doi: 10.1111/j.1399-0039.1996.tb02509.x |
| | U.S. Pat. No. 10,730,944B2 |
| | U.S. Pat. No. 11,739,150B2 |
| IL-6 | Ehsani, P et al. “Expression of anti human IL-4 and IL-6 scFvs in transgenic |
| | tobacco plants.” Plant molecular biology vol. 52, 1 (2003): 17-29. |
| | doi: 10.1023/a: 1023902407855 |
| IL-4 | Ehsani, P et al. “Expression of anti human IL-4 and IL-6 scFvs in transgenic |
| | tobacco plants.” Plant molecular biology vol. 52, 1 (2003): 17-29. |
| | doi: 10.1023/a: 1023902407855 |
| | Spangler, Jamie B et al. “A strategy for the selection of monovalent antibodies |
| | that span protein dimer interfaces.” The Journal of biological chemistry vol. |
| | 294, 38 (2019): 13876-13886. doi: 10.1074/jbc.RA119.009213 |
| | Obmolova, Galina et al. “Induced conformational change in human IL-4 upon |
| | binding of a signal-neutralizing DARPin.” Proteins vol. 83, 6 (2015): 1191-7. |
| | doi: 10.1002/prot.24815 |
| | U.S. Pat. No. 11,453,727B2 |
| IL-10 | Josephson, Kristopher et al. “Noncompetitive antibody neutralization of IL-10 |
| | revealed by protein engineering and x-ray crystallography.” Structure (London, |
| | England: 1993) vol. 10, 7 (2002): 981-7. doi: 10.1016/s0969-2126(02)00791-8 |
| IL-11 | US20180186871A1 |
| IL-13 | Ultsch, Mark et al. “Structural basis of signaling blockade by anti-IL-13 |
| | antibody Lebrikizumab.” Journal of molecular biology vol. 425, 8 (2013): |
| | 1330-9. doi: 10.1016/j.jmb.2013.01.024 |
| | Rothenberg, Marc E et al. “Intravenous anti-IL-13 mAb QAX576 for the |
| | treatment of eosinophilic esophagitis.” The Journal of allergy and clinical |
| | immunology vol. 135, 2 (2015): 500-7. doi: 10.1016/j.jaci.2014.07.049 |
| | US20110086038 |
| | Kasaian, Marion T et al. “Efficacy of IL-13 neutralization in a sheep model of |
| | experimental asthma.” American journal of respiratory cell and molecular |
| | biology vol. 36, 3 (2007): 368-76. doi: 10.1165/rcmb.2006-0244OC |
| | Teplyakov, Alexey et al. “Epitope mapping of anti-interleukin-13 neutralizing |
| | antibody CNTO607.” Journal of molecular biology vol. 389, 1 (2009): 115-23. |
| | doi: 10.1016/j.jmb.2009.03.076 |
| | US11453727B2 |
| IL-20 | Jain, Tushar et al. “Biophysical properties of the clinical-stage antibody |
| | landscape.” Proceedings of the National Academy of Sciences of the United |
| | States of America vol. 114, 5 (2017): 944-949. doi: 10.1073/pnas.1616408114 |
| | US20080311115 |
| IL-22 | US20130005033 |
| IL-33 | US20180346562 |
| | Park, Soo Bin et al. “Blocking of the IL-33/ST2 Signaling Axis by a Single- |
| | Chain Antibody Variable Fragment (scFv) Specific to IL-33 with a Defined |
| | Epitope.” International journal of molecular sciences vol. 21, 18 6953. 22 Sep. |
| | 2020, doi: 10.3390/ijms21186953 |
| IFNα | US20150246130 |
| | US20070014724 |
| | Maurer, Brigitte et al. “Structural basis of the broadly neutralizing anti- |
| | interferon-α antibody rontalizumab.” Protein science: a publication of the |
| | Protein Society vol. 24, 9 (2015): 1440-50. doi: 10.1002/pro.2729 |
| | Ouyang, Songying et al. “Structural insights into a human anti-IFN antibody |
| | exerting therapeutic potential for systemic lupus erythematosus.” Journal of |
| | molecular medicine (Berlin, Germany) vol. 90, 7 (2012): 837-46. |
| | doi: 10.1007/s00109-012-0866-3 |
| IFNγ | Bourne, Philip C et al. “Three-dimensional structures of a humanized anti-IFN- |
| | gamma Fab (HuZAF) in two crystal forms.” Acta crystallographica. Section |
| | D, Biological crystallography vol. 60, Pt 10 (2004): 1761-9. |
| | doi: 10.1107/S0907444904018670 |
| | Pini, A et al. “Design and use of a phage display library. Human antibodies |
| | with subnanomolar affinity against a marker of angiogenesis eluted from a |
| | two-dimensional gel.” The Journal of biological chemistry vol. 273, 34 (1998): |
| | 21769-76. doi: 10.1074/jbc.273.34.21769 |
| | Froyen, G et al. “Effect of VH and VL consensus sequence-specific primers on |
| | the binding and neutralizing potential of a single-chain FV directed towards |
| | HuIFN-gamma.” Molecular immunology vol. 32, 7 (1995): 515-21. |
| | doi: 10.1016/0161-5890(95)00010-c |
| | Fan, Z C et al. “Comparison of the three-dimensional structures of a |
| | humanized and a chimeric Fab of an anti-gamma-interferon antibody.” Journal |
| | of molecular recognition: JMR vol. 12, 1 (1999): 19-32. |
| | doi: 10.1002/(SICI)1099-1352(199901/02)12: 1 < 19::AID-JMR445 > 3.0.CO; 2-Y |
| CXCL12 | Zhong, Cuiling et al. “Development and preclinical characterization of a |
| | humanized antibody targeting CXCL12.” Clinical cancer research: an official |
| | journal of the American Association for Cancer Research vol. 19, 16 (2013): |
| | 4433-45. doi: 10.1158/1078-0432.CCR-13-0943 |
| | US20110206660 |
| | US20110117113 |
| | US20100092460 |
| | Claes, Katrien et al. “Modular Integrated Secretory System Engineering in |
| | Pichia pastoris To Enhance G-Protein Coupled Receptor Expression.” ACS |
| | synthetic biology vol. 5, 10 (2016): 1070-1075. doi: 10.1021/acssynbio.6b00032 |

The bispecific antibodies of the present disclosure can be made using any method known in the art. One approach for single cell manufacturing of bispecific antibodies is to use antibodies that are based on the same light chain in combination with a minimal set of Fc mutations to drive heavy chain heterodimerization. The resulting bispecific antibodies can readily be purified from the two undesired byproducts. As used herein the term “common light chain” refers to an immunoglobulin light chain comprising a variable domain that can productively associate with multiple heavy chain variable domains to form a paratope with each heavy chain variable region that is capable of specifically binding the epitope bound by the antibody in which the heavy chain variable region was originally encountered. Various antibody heavy or light chains, or portions thereof, are referred to herein as being “related” to the corresponding chain or portion thereof of an enumerated or specifically identified antibody. This denotes that the chain or portion thereof has identical sequence to the corresponding chain or portion thereof of an enumerated or specifically identified antibody, save for specifically indicated modifications, such as amino acid substitutions, of which there may be none.

Once administered, the biological activity of the bispecific antibody, or antigen-binding fragments thereof, can be measured by any suitable method known in the art. For example, the biological activity can be assessed by determining the stability of the bispecific antibody. The biological activity of the bispecific antibody also can be assessed by determining its binding affinity to antigen-containing peptides and/or by assessing its binding affinity to peptides with which it may cross-react. The term “affinity” refers to the equilibrium constant for the reversible binding of two agents and is expressed as the dissociation constant (K_{D}). Affinity of a binding agent to a ligand, such as affinity of an antibody for an epitope, can be, for example, from about 1 femtomolar (fM) to about 1 millimolar (mM) (e.g., from about 1 picomolar (pM) to about 1 nanomolar (nM), or from about 1 nM to about 1 micromolar (μM)). Antibody affinity for an antigen or epitope of interest can be measured using any art-recognized assay. Such methods include, for example, fluorescence activated cell sorting (FACS), separable beads (e.g., magnetic beads), antigen panning, and/or ELISA (see, e.g., Janeway et al. (eds.), Immunobiology, 5th ed., Garland Publishing, New York, N.Y., 2001).

### 4. COMPOSITIONS

Further disclosed herein are compositions suitable for use in the disclosed methods. The compositions may comprise a bispecific binding molecule configured to specifically bind a cancer cell membrane-associated target and one of an anti-inflammatory cytokine or chemokine, a tumorigenic cytokine or chemokine, or a T cell activator. In some embodiments, the compositions comprise a bispecific binding molecule configured to specifically bind: Siglec15 (S15); and one of an anti-inflammatory cytokine or chemokine, a tumorigenic cytokine or chemokine, or a T cell activator, as described above. The descriptions of the bispecific molecules provided above, are applicable to the compositions described herein.

The compositions may comprise excipients or pharmaceutically acceptable carriers. The choice of excipients or pharmaceutically acceptable carriers will depend on factors including, but not limited to, the particular mode of administration, the effect of the excipient on solubility and stability, and the nature of the dosage form. The compositions of the present invention will be readily apparent to those skilled in the art. Techniques and formulations may be found, for example, in Remington's Pharmaceutical Sciences, 19th Edition (Mack Publishing Company, 1995).

The term “pharmaceutically acceptable carrier,” as used herein, means a non-toxic, inert solid, semi-solid or liquid filler, diluent, encapsulating material, surfactant, cyclodextrins or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; tale; excipients such as, but not limited to, cocoa butter and suppository waxes; oils such as, but not limited to, peanut oil, cottonseed oil, safflower oil, sesame oil, olive oil, corn oil and soybean oil; surfactants such as, but not limited to, cremophor EL, cremophor RH 60, Solutol HS 15 and polysorbate 80; cyclodextrins such as, but not limited to, alpha-CD, beta-CD, gamma-CD, HP-beta-CD, SBE-beta-CD; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as releasing agents, coating agents, preservatives and antioxidants can also be present in the composition, according to the judgment of the formulator.

The route by which the disclosed compounds are administered and the form of the composition will dictate the type of carrier to be used. The composition may be in a variety of forms, suitable, for example, for systemic administration (e.g., oral, rectal, nasal, sublingual, buccal, implants, or parenteral injections) or topical administration (e.g., dermal, pulmonary, nasal, aural, ocular, liposome delivery systems, or iontophoresis).

Any of the above compositions or formulations disclosed herein may further comprise at least one additional therapeutic agent. In some embodiments, the at least one additional therapeutic agent comprises at least one chemotherapeutic agent.

### 5. KITS

Also disclosed herein are kits comprising a bispecific binding molecule (e.g., compositions, formulations, or medicaments thereof) as disclosed herein.

The kits can also comprise other agents (e.g., chemotherapeutic agents) and/or products co-packaged, co-formulated, and/or co-delivered with other components. The kits can also comprise instructions for using the components of the kit. The instructions are relevant materials or methodologies pertaining to the kit. The materials may include any combination of the following: background information, list of components, brief or detailed protocols for using the bispecific binding molecule, troubleshooting, references, technical support, and any other related documents. Instructions can be supplied with the kit or as a separate member component, either as a paper form or an electronic form which may be supplied on computer readable memory device or downloaded from an internet website, or as recorded presentation.

It is understood that the disclosed kits can be employed in connection with the disclosed methods. The kit may further contain containers or devices for use with the methods disclosed herein. For example, the kits can comprise delivery devices (e.g., syringes).

The kits provided herein are in suitable packaging. Suitable packaging includes, but is not limited to, vials, bottles, jars, flexible packaging, and the like. Individual member components of the kits may be physically packaged together or separately.

### 6. EXAMPLES

### Materials and Methods

Bispecific antibody design and production. Various antibodies were engineered including the bispecific antibody(BsAb) that binds both Siglec15 (S15) and TGF-β (abbreviated ST herein), an IgG mAb that binds S15 only (abbreviated S herein), and an IgG mAb that binds TGF-β only (abbreviated T herein). The bispecific antibody targeting both S15 and TGFβ (e.g., anti Siglec15 and anti-TGFβ) was developed using the antibody clones 1H3) Linda Liu, Benjamin Dallas Flies, Solomon Langermann, 2017) and 2G750 variable region sequences by incorporating OrthoMab point mutations into the Fab interface that enable >90% correct pairing of the two antibodies. The amino acid sequences were converted to human codon optimized DNA sequence with Fab domains in the BsAb separated by a flexible (GGGGS)₆ (SEQ ID NO: 1)linker in the IgG-Fab format. The DNA was synthesized through Twist biosciences and cloned into mammalian expression vector paH for secretion in Expi293 cells. The BsAb heavy and light chain DNA was transfected in a 1:2 ratio using manufacturer protocols using expifectamine (Thermofisher) and the supernatant was collected after the cell viability dropped below 60% and purified using Protein-A/G affinity chromatography using FPLC and purity was verified by SDS-PAGE.

Anti-S15 light chain:

(SEQ ID NO: 2)
DIQMTQSPSSLSASVGDRVTITCKASDHINNWLAWYQQKPGKAPKLLISG
ATSLETGVPSRFSGSGSGTDYTFTISSLQPEDIATYYCQQYWSSPLTFGG
GTKVEIK.

Anti-S15 heavy chain:

(SEQ ID NO: 3)
QVQLQESGPGLVKPSETLSLTCTVSGFSLSNYGVHWVRQPPGKGLEWIVL
IWSDGSTTYNSALKSRVTISKDTSKNQVSLKLSSVTAADTAVYYCARHPY
DDYSGYYYTMDYWGQGTLVTVSS.

Binding affinity measurement. The binding affinity of the 1H3-2G7 BsAb was measured using MST to both Siglec15 (Acrobiosystems) and TGFβ (Acrobiosystems) to ensure that there was no significant change in binding compared to the parent mAbs 1H3 and 2G7. Briefly the antibodies were fluorescently labeled using the Red-maleimide 2nd generation kit (Nanotemper) and 10 μl of antibody was mixed with 10 μl of 16 serial 2 fold dilutions of either S15 or TGFβ for the appropriate antibody, both proteins and Abs diluted in a PBS-Tween buffer (PBS with 0.05% Tween 20). The pre-mixed samples were subsequently loaded into capillaries, and the thermophoresis of each sample was measured using Red laser at medium MST power on Monolith NT.115 (Nanotemper). The binding affinities were calculated using a 1:1 binding fit.

Cell line and cell culture. Mouse 4T1 TNBC was stably transfected with a vector carrying the mCherry red fluorescent protein (RFP), firefly luciferase (Luc) and the puromycin resistance gene, followed by selection in the presence of puromycin. The 4T1 RFP/Luc cells were maintained in Dulbecco's Modified Eagle Medium: Nutrient Mixture F-12 (DMEM/F12) supplemented with 10% fetal bovine serum (FBS) (Gibco) and 1% Penicillin/Streptomycin at 37° C. and 5% CO₂ in a humidified atmosphere.

4T1 TNBC orthotopic model. Six-week-old female BALB/c mice were purchased from Charles River Laboratories and maintained under pathogen-free conditions. All animal-handling procedures were approved by the Institutional Animal Care and Use Committee at University of North Carolina at Chapel Hill. To establish the orthotopic tumor model, 106 4T1 TNBC cells suspended in PBS were injected at the fourth nipple of each mice, and tumor growth was monitored every 2 days by caliper and calculated as (L×W2)/2, where “L” represents the longer dimension and “W” represents the shorter one. Treatments were initiated on Day 14 and administered a total of 3 times every other day. Before the first treatment, mice were randomized into different groups (n=8-10 per group) receiving the following interventions: control Ab, ST bsAb, S+ T mAb, S mAb only, and T mAb only.

Flow cytometry assay. Tumor-infiltrating immune cells were analyzed by flow cytometry. In brief, tumor tissues were harvested and digested with collagenase A and DNase at 37° C. for 60 min. After lysis of red blood cells, cells were resuspended in 5 mL of PBS. 2×10⁶ cells were stained with fluorescently labeled antibodies for surface marker expression analysis. After staining, cells were fixed with 300 μL 4% PFA and analyzed via FACS (BD LSR II).

To access cytokine production by tumor-infiltrating cells, cells were stained with surface marker, washed, fixed, and permeabilized using the cytofix/cytoperm kit from BD Pharmingen, according to manufacturer's instructions. Intracellular stains were performed using anti-IFN-gamma antibody. Cells were washed and analyzed on an 18-color flow cytometer (LSR II). Data were analyzed with FlowJo 8.6 software (TreeStar).

Immunofluorescence staining. At the endpoint of the study, the mice were sacrificed and tumors were harvested and prepared for immunohistology staining. After deparaffmizing, antigen retrieval, and permeabilization, tissue sections were blocked in 1% bovine serum albumin (BSA) at room temperature for 1 h. Primary antibodies conjugated with fluorophores (BD, eBioscience, abeam) were incubated overnight at 4° C., and nuclei were counterstained with DAPI-containing mounting medium (Vector Laboratories Inc., Burlingame, CA). All antibodies were diluted according to the manufacturer's manual. Images were taken using fluorescence microscopy (Nikon, Tokyo, Japan).

Luminex Assay. A multiplex analysis kit (Cytokine & Chemokine Convenience 26-Plex Mouse ProcartaPlex™ Panel 1) for 26 chemokines and cytokines was obtained from Invitrogen (Waltham, MA). Tumor samples were homogenized by hard tissue homogenizing mix with bead homogenizer, then the assay performed according to the manufacturer's protocol. Data was collected using the Luminex-MAGPIX® system (Luminex, Austin, TX). Data analysis was performed using the xPONENT software package. (Luminex, Austin, TX). Five-parameter regression was used to calculate the sample concentrations from the standard curves.

H&E staining. The tumors were fixed in 4% formalin, paraffin-embedded and sectioned for hematoxylin and eosin (H&E) staining. Metastasis were determined by H&E staining and photographed by optical microscopy.

Statistical analysis. All data are presented as mean±SD. GraphPad Prism V.8 software was used to generate graphs and perform statistical analyses. Either Student's t-test or two-way analysis of variance (ANOVA) was used to determine statistically significant differences between treatment groups. Survival analysis was performed using the Kaplan-Meier method with a log-rank test to determine statistical significance. All p values less than 0.05 were considered statistically significant.

### Example 1

### S15 and TGF-β are Both Upregulated in Human Breast Cancer Patients

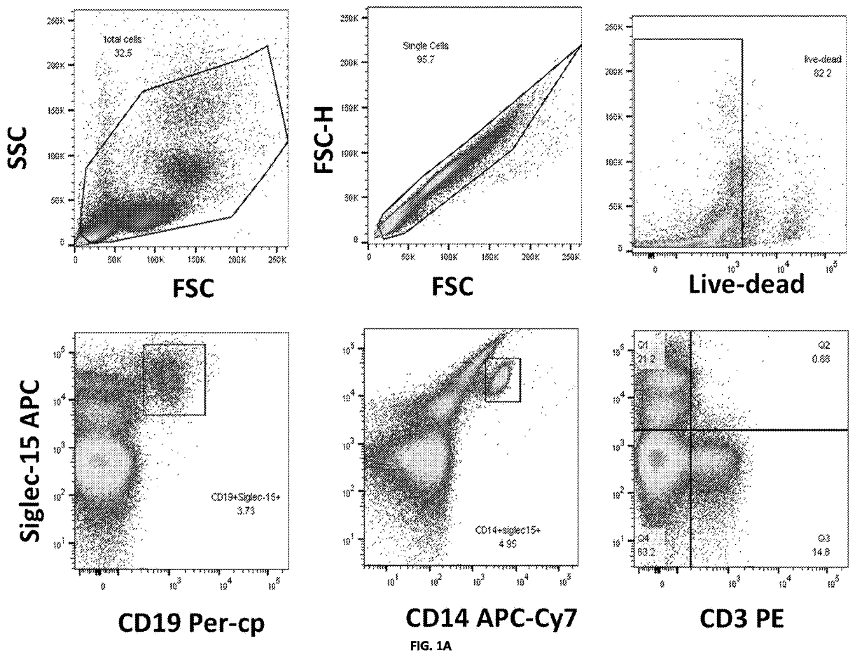

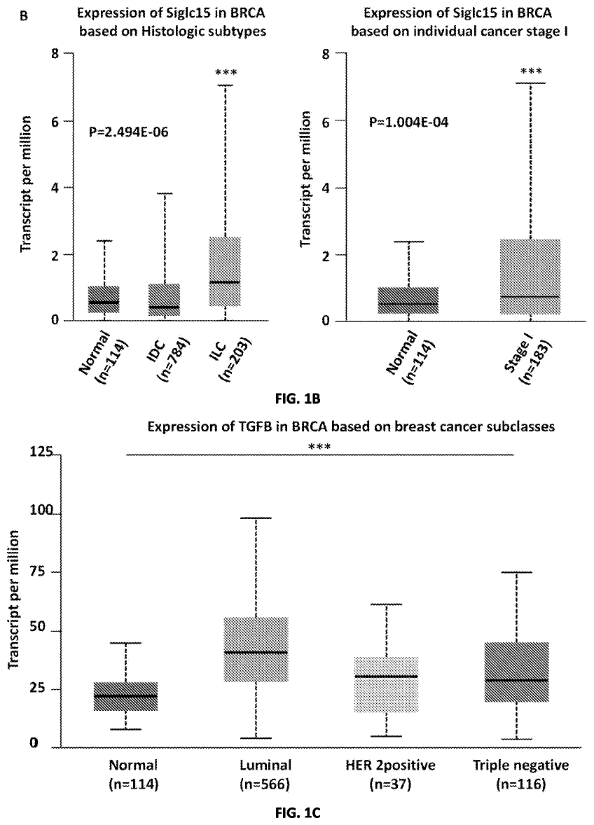

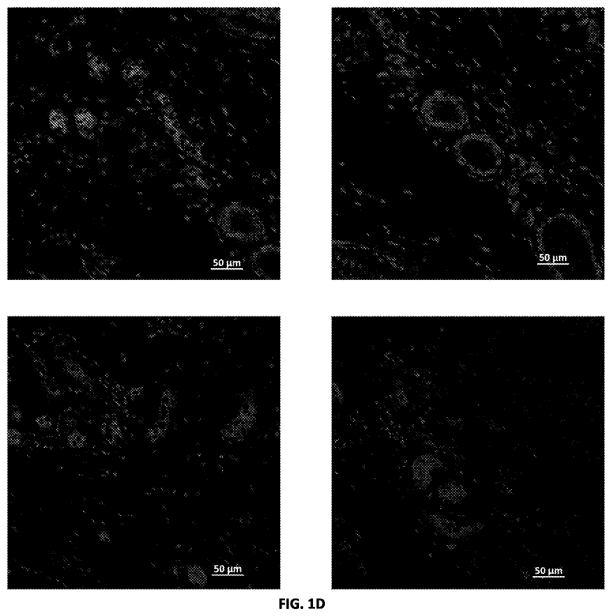

S15 expression was detected only on a subset of macrophages and B cells, but not in T-cells (FIG. 1A). Next, utilizing the TCGA data bank, it was confirmed that S15 (FIG. 1B) and TGF-β (FIG. 1C) were upregulated in breast cancer tissue compared to normal tissue, the latter in both HER2-positive and TNBC tissues. Histology on tumor specimens from TNBC patients consistently detected S15 expression (FIG. 1D). These results confirmed S15 is upregulated in breast cancer tissues.

### Example 2

### Characterization of S15/TGF-β bsAb, Anti-S15 and Anti-TGF-β mAbs In Vitro

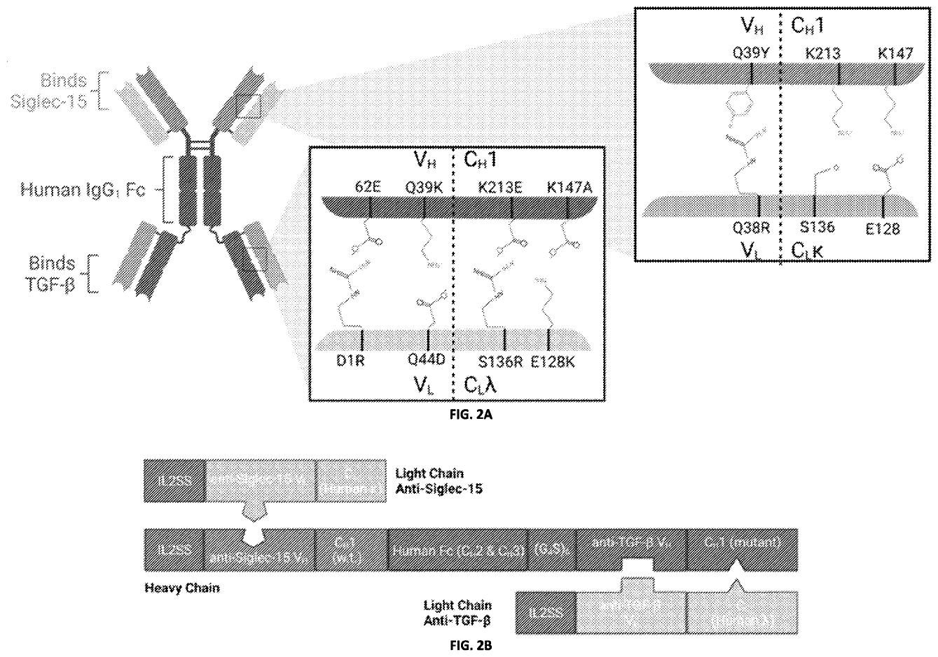

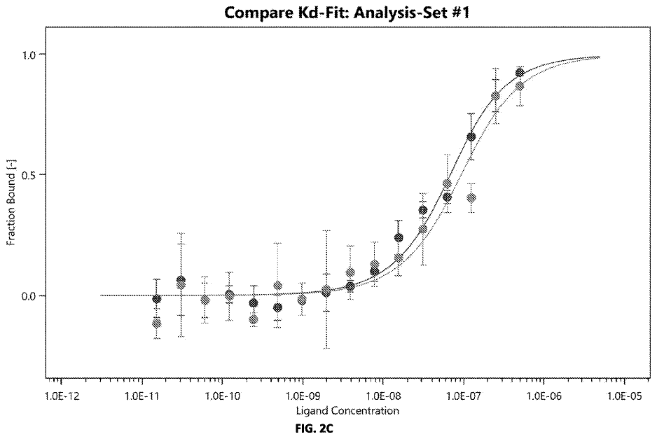

To preserve the bivalent binding of S15, an IgG-Fab design was utilized to construct the S15/TGF-β3 bsAb, utilizing the OrthoMab platform that ensures proper pairing of distinct heavy and light chains by introducing a small number of site-specific amino acid mutations in both the heavy chain and light chain at the CH1 interface. S15/TGF-β bsAb displayed the expected size and was able to bind S15 and TGF-β with comparable affinity as the individual parent mAbs (FIG. 2).

### Example 3

### ST bsAb Binds 4T1 Cells and Tumor Tissues from TNBC Patients Ex Vivo

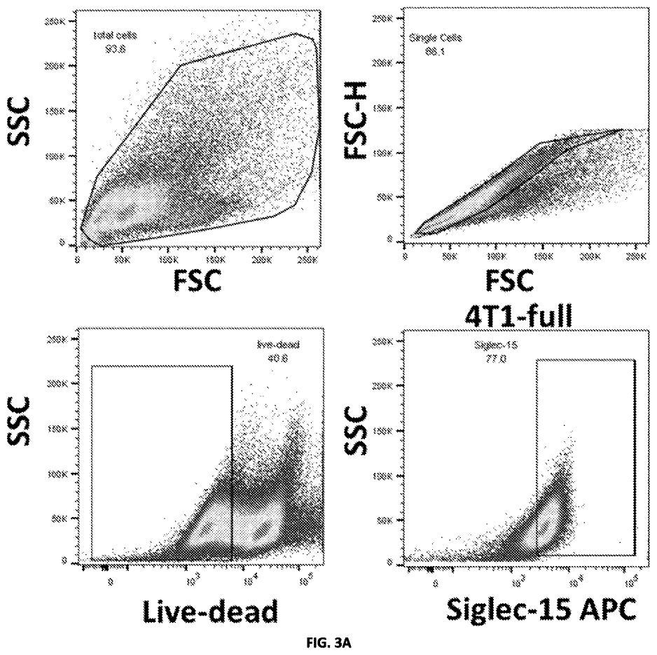

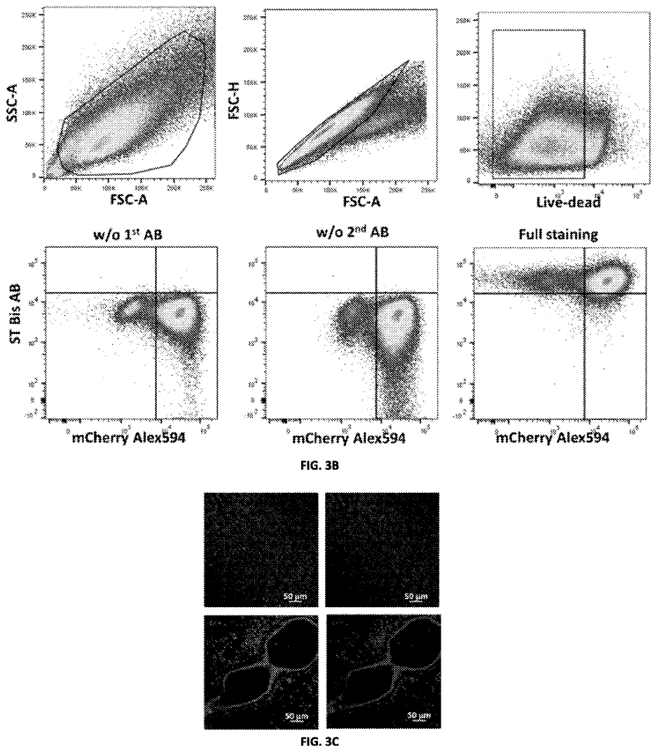

To ascertain the suitability of the murine 4T1 TNBC model, the ability of ST bsAb to bind 4T1 TNBC cells was ascertained. Via flow cytometry, high levels of S15 expression were detected on 4T1 cells in vitro, labeled an anti-S15 mAb (FIG. 3A). To confirm the activity of the ST bsAb, 4T1 cells were isolated from mice bearing orthotopic 4T1 tumors, and it was found that ST bsAb effectively bound the isolated 4T1 cells (FIG. 3B). Histology on tumor tissues collected from the 4T1 tumor bearing mice determined that engineered ST bsAb bound to tumor tissue.

### Example 4

### ST bsAb Inhibited the Growth of Established 4T1 TNBCs in Mice

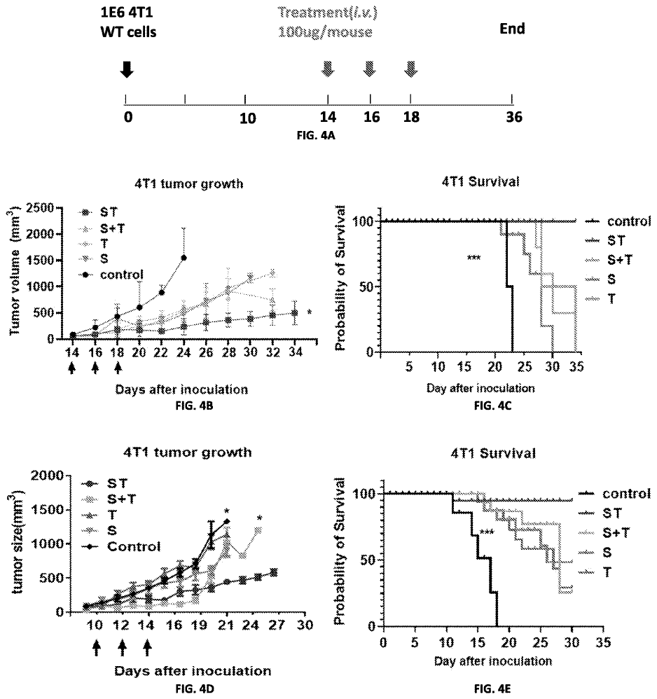

The efficacy of ST bsAb therapy was assessed in 4T1 triple negative breast cancer murine model. On Day 14 following inoculation of the 4T1 tumors, mice were given various treatments, administered a total of 3 times given every 2 days. The treatments included the ST bsAb, a control mAb, a mAb against S15 only (S), a mAb against TGF-β only (T), and a mAb cocktail of both S and T(S+T). Clear evidence of tumor suppression was observed with all of the non-control mAbs, with apparent anti-tumor activity following the very first of the three doses. The overall anti-tumor activity of S, T, and S+ T were similar across the entire duration of the study, with slower but sustained growth of the tumor, and resulted in similar overall survival, with all mice passing the point necessitating humane sacrifice on Day 34. In contrast to these interventions, the ST bsAb treatment effectively inhibited tumor growth (FIGS. 4B and 4D), with no statistically significant difference in the tumor size from the last day of the treatment (Day 18) through the end of the study (Day 36). All mice receiving the ST bsAb-treatment survived more than 30 days (FIGS. 4C and 4E). In addition, no metastasis was detected in lung and less metastasis in liver of all mice treated ST bsAb, while metastases were detected in mice treated with control mAb, S+ T, S, and T.

### Example 5

### ST bsAb Modulated the Immune-Suppressive TME

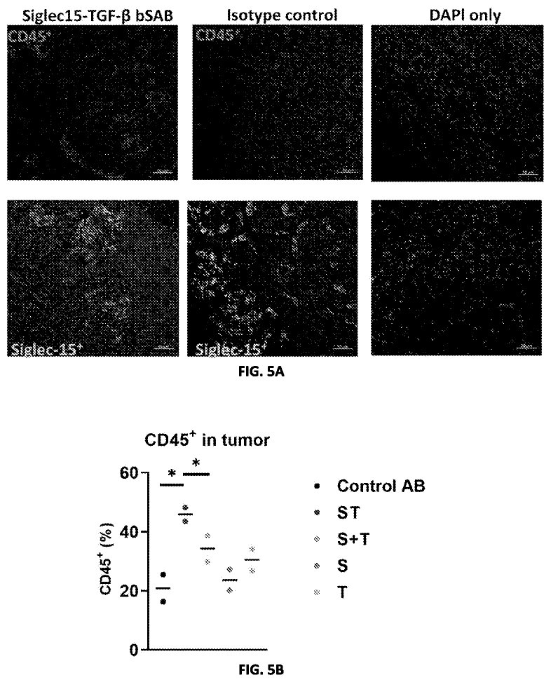

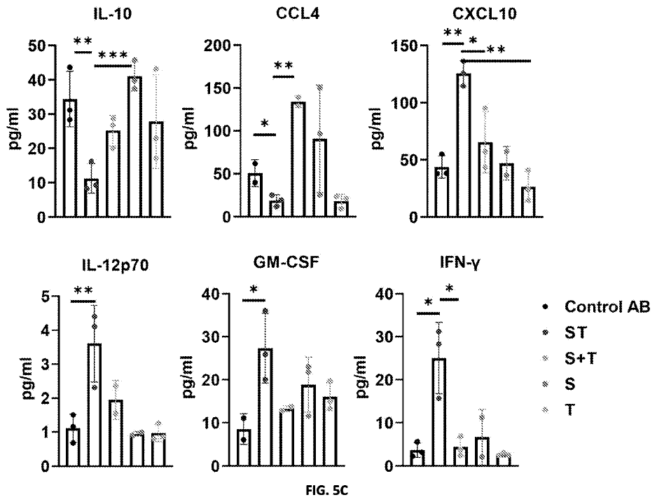

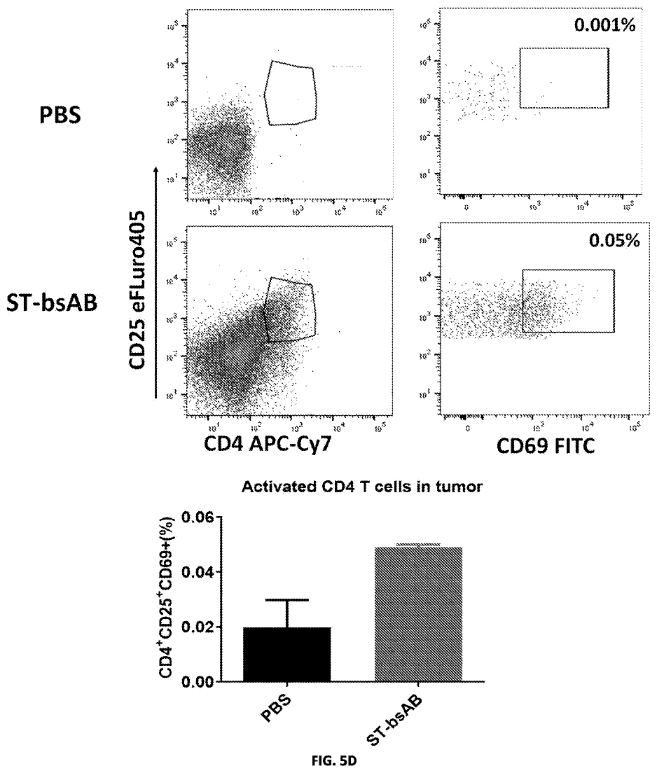

Tumor growth is determined not only by intrinsic tumor biology, but also by the immune microenvironment within the tumor. To determine what factors may substantiate the more effective tumor growth suppression observed with the ST bsAb, it was assessed whether the treatment altered specific immunological aspects of the TME. CD45 is an important regulator of T- and B-cell antigen receptor signaling; activated, memory and cytotoxic tumor-infiltrating T-lymphocytes all express CD45 on their cell surface. A marked increase in the CD45+ cells was consistently detected in tumors of mice treated with ST, as revealed by both staining of tumor sections (FIG. 5A) as well as flow cytometry analysis of tumor-derived single cell suspensions from different treatment groups (FIG. 5B).

The TME is also characterized by the secretion of diverse cytokines in different types and stages of cancers, capable of either inhibiting tumor growth or contributing to chronic inflammation that supports tumor growth in both autocrine and paracrine manners leading to poor cancer outcomes. The change in cytokines in the TME was assessed following treatment with different mAbs. Consistent with the enhanced tumor suppression following ST bsAb treatment, the level of proinflammatory cytokines such as IL-10 and CCL4 that inhibit anti-tumor immunity was significantly decreased (FIG. 5C). The chemokine CXCL10 is a seven trans-membrane-spanning G protein-coupled receptor predominantly expressed on activated T lymphocytes (Th1), natural killer (NK) cells, inflammatory dendritic cells, macrophages, and B cells and serves as a chemoattractant for monocytes, T cells and NK cells. Increased CXCL10 was found in mice treated with ST bsAb (FIG. 5C), and increased activated CD4 T cells (FIG. 5D). GM-CSF is essential for the differentiation of dendritic cells responsible for processing and presenting tumor antigens for the priming of antitumor cytotoxic T lymphocytes. IL-12 regulates inflammation by linking innate and adaptive immune responses, generally through increased secretion of interferon-γ (IFN-γ) that promote Th1 cell immunity and stimulate M1 macrophage as part of an overall activation of anti-tumor immune response. Consistent with reduced immune suppression, treatment with ST bsAb significantly enhanced GM-CSF secretion, increased IL-12p70, and increased IFN-γ levels in the TME, underscoring the effectiveness of the ST bsAb in mediating a robust, multifaceted anti-tumor response.

### Example 6

### ST bsAb Enhanced Systemic T Cell Activation

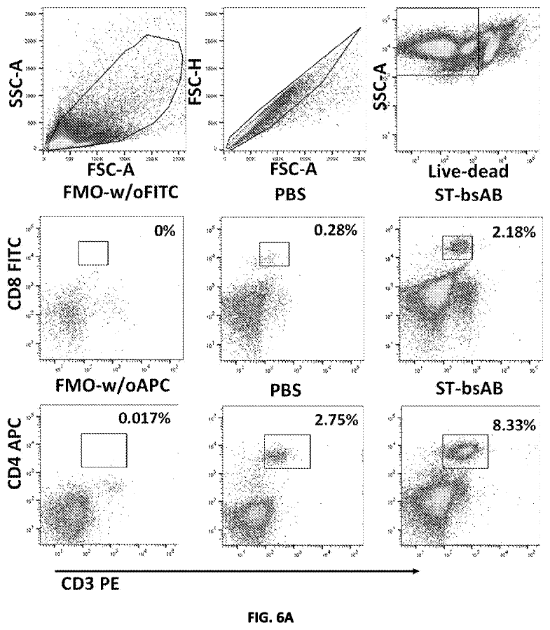

Recent studies have shown that systemic activation of T cells, reflected by patients with higher numbers of circulating CD4+ and CD8+ T-cells, was associated with an overall survival benefit compared to patients with low number of circulating CD4+ T-cells. The number of CD4+ and CD8+ T-cells in lymph nodes from tumor bearing mice was analyzed following different treatments. Following ST bsAb treatment, markedly more CD4⁻ and CD8⁻ T-cells were detected, including activated CD4+ and CD8+ T-cells (FIG. 6C). These results are consistent with ST bsAb mediating an active, ongoing immune response in 4T1-TNBC bearing mice.

### Example 7

### ST bsAb Appeared to Reduce 4T1 Metastasis

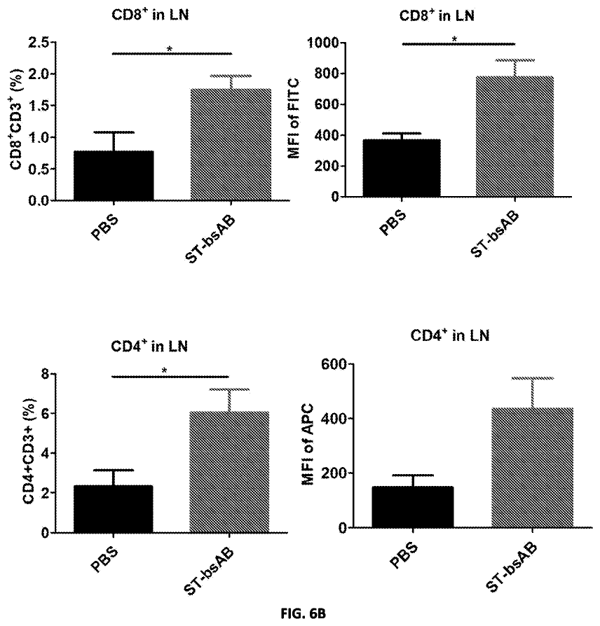

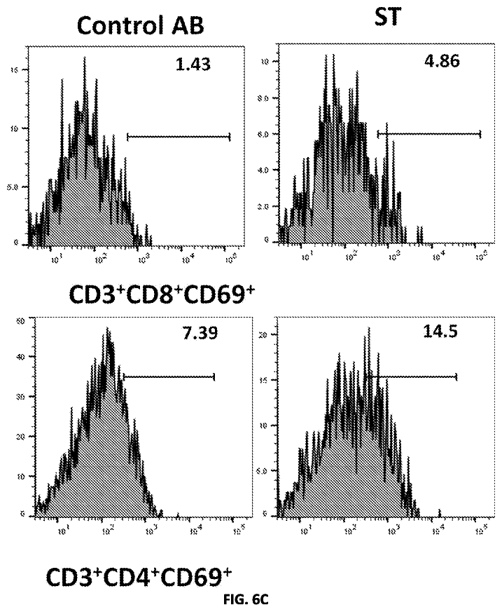

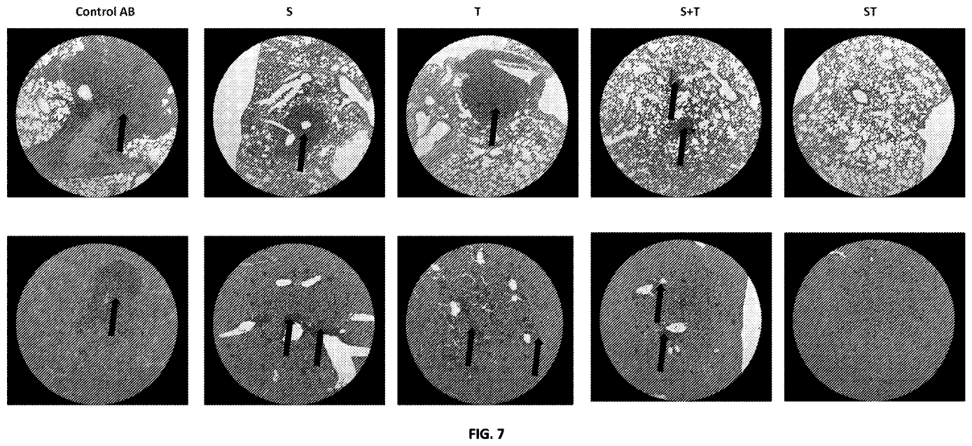

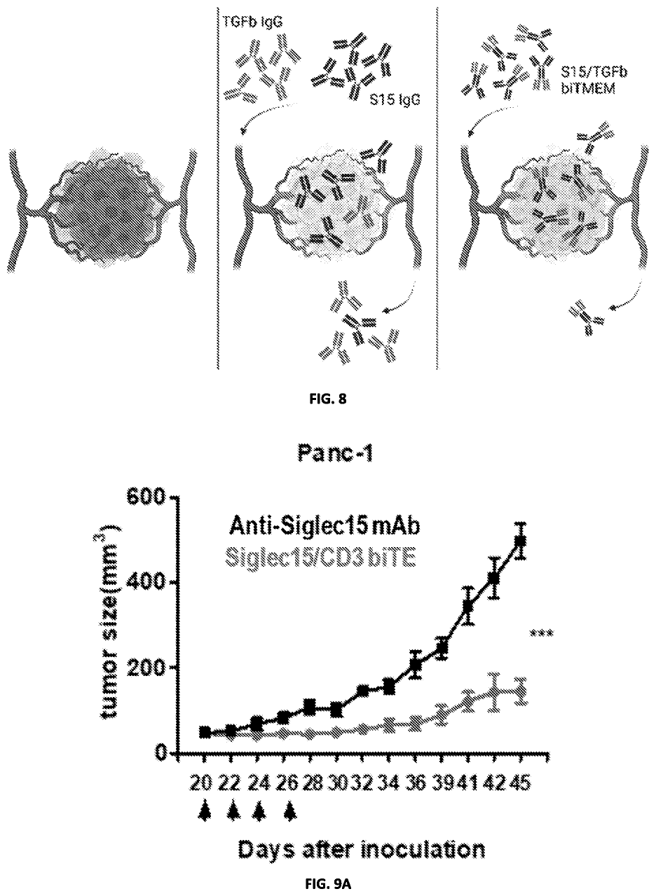

Given the promising results above, it was further evaluated whether the ST bsAb-mediated enhanced T-cell activity was able to impact TNBC metastasis, commonly found in the liver in spleen. Surprisingly, the enhanced T-cell activity appeared to be sufficient to eliminate 4T1 metastasis as metastasis was unable to be detected in either the lung or the liver of mice treated with the ST bsAb, whereas metastasis was found in mice from all other treatment groups (FIG. 7).

### Example 8

### S15/CD3 biTE Suppresses Pane-1 Tumor in NSG Mouse Infused with Human PBMCs, Increases T Cell Infiltration in Tumor

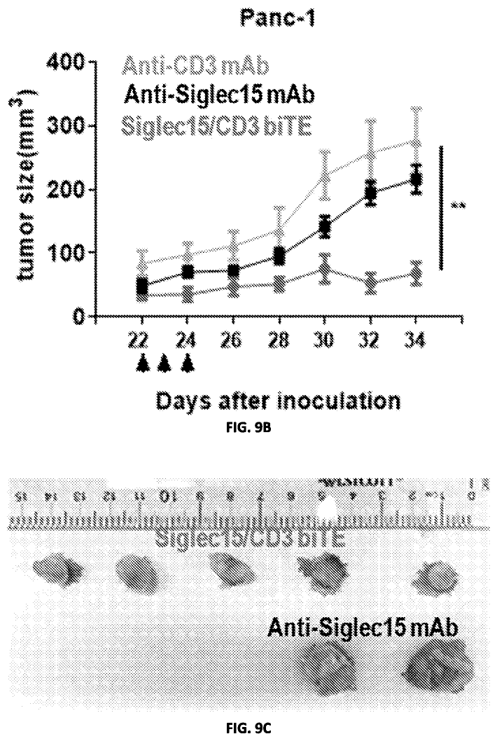

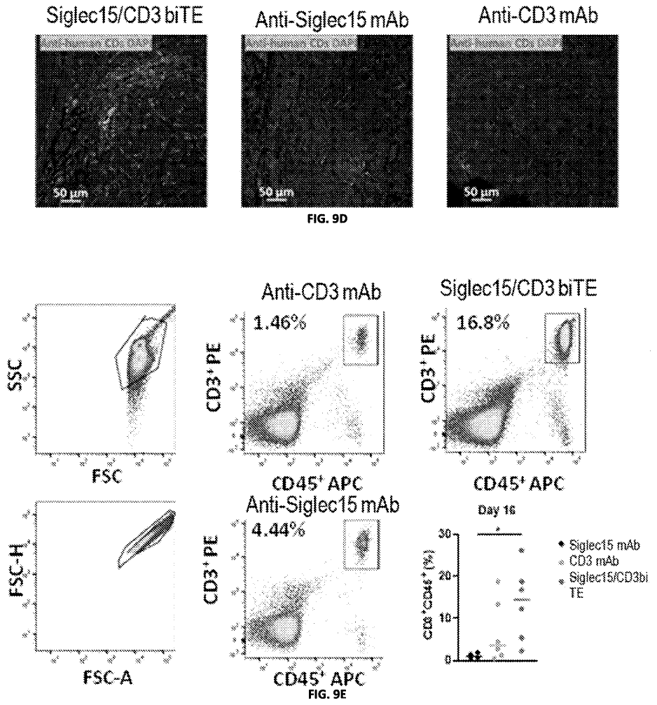

A S15/CD3 bispecific T cell engager (BiTE) in the IgG-Fab format was engineered and evaluated for efficacy in a subcutaneous human Panc-1 pancreatic tumor model. Various mAbs were dosed into mice by IV injection, followed by human PBMCs, and the size of the tumors were assessed over time. S15/CD3 biTE treatment effectively limited tumor growth over time, in marked contrast to tumors in mice receiving anti-S15 mAb (FIG. 9A). To ensure reproducibility, a second study was conducted, and also included an anti-CD3 mAb control. While ongoing, S15/CD3 biTE effectively limited tumor growth, in marked contrast to tumors in mice receiving either anti-S15 or anti-CD3 mAbs (FIG. 9B). The improved efficacy appears to directly correlate with substantial increase in T-cells, both in the tumor (as validated by histology, FIG. 9D) and in the circulation (as validated by flow cytometry, FIG. 9E). The substantial increase in T-cells was consistently observed in different mice treated with S15/CD3 biTE. While not yet tested against TNBC, these results verified the ability to engineer a functional S15-targeted biTE for T-cell therapy, as exemplified in the S15/CD3 biTE for immunotherapy.

### Example 9

### Design and Characterization of S15/CD3 biTE

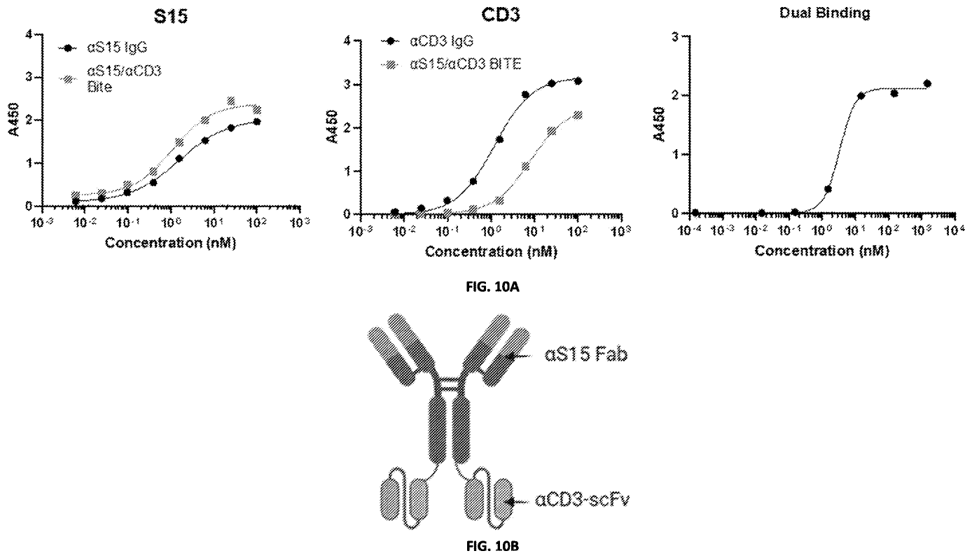

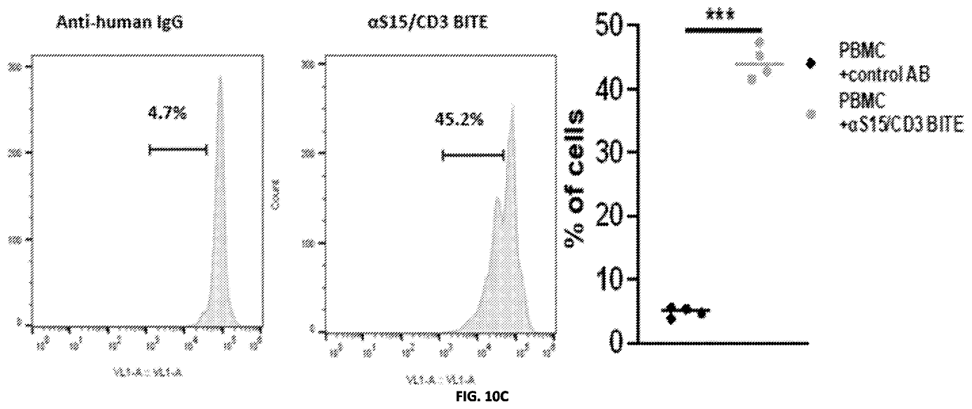

The bispecific S15/CD3 biTE molecule was engineered in the IgG-scFv format (FIG. 10B). Each molecule consists of symmetric heavy chains dimerized via human IgG1-Fc, resulting in 2 Fabs against S15 and 2 scFvs against CD3. The binding affinities to S15 and CD3 were assessed via ELISA. The EC₅₀ of STAB against S15 was highly comparable to the EC₅₀ found with the parent IgG. The binding affinity of STAB against CD3 was slightly reduced compared to the parent IgG, but remained in the single-digit nanomolar range reflective of strong affinity to CD3. The simultaneous binding to both CD3 and S15 was also confirmed (FIG. 10A). To further confirm CD3 binding, BiTE or control IgG was incubated with CD3+ T cells in PBMCs previously stained with carboxyfluorescein (CFSE) diacetate, and then analyzed using flow cytometry. Compared to a non-specific IgG control, BiTE induced a significantly higher abundance of T cells within PBMCs, increasing the fraction of T cells by nearly 10-fold (FIG. 10C). This increase in T cells was consistently observed across PBMCs from three different donors. These results suggest that this biTE can significantly enhance the proliferation of T cells, likely by activating CD3+ T cells.

### Example 10

### S15/CD3 biTE Promotes Efficient Killing of S15+PDAC and NSCLC Cells in Co-Culture Studies

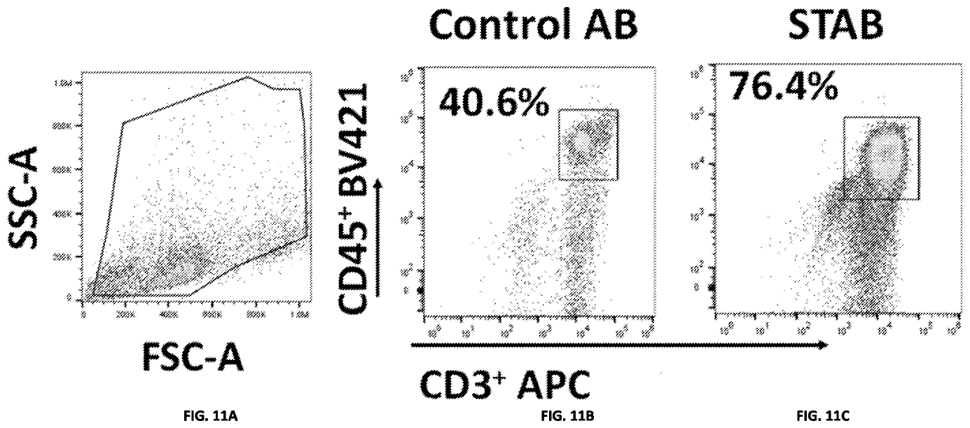

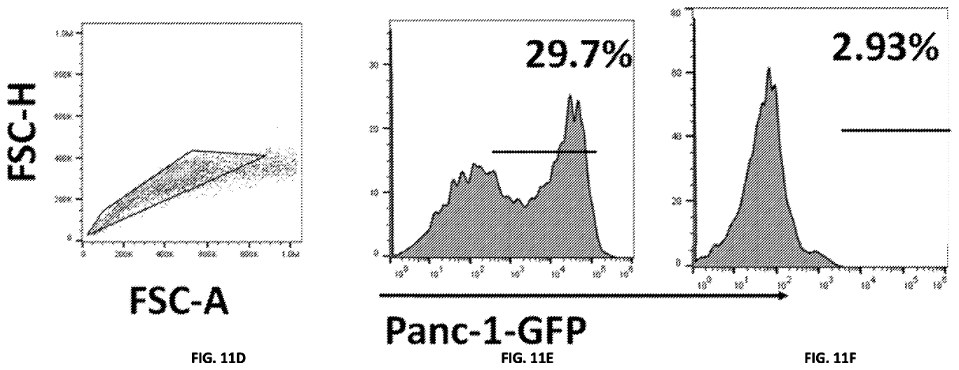

Next, it was investigated whether S15/CD3 biTE can enhance T cell mediated killing of specific cancer cells. Panc-1 human pancreatic cancer cells expressing GFP were mixed with human PBMCS at a ratio of 3 PBMCS to 1 cancer cell, followed by addition of either biTE or control IgG. In Panc-1/PBMC co-cultures treated with control IgG, there was a substantial cancer cell population consistent with a lack of cancer cell killing (FIG. 11A). In contrast, the addition of biTE markedly reduced the Panc-1 population. The marked reduction of cancer cells is correlated with greater abundance of T cells (FIG. 11B). Anti-tumor activity against H460 NSCLC cells expressing mCherry was also verified. In similar co-culture studies, biTE reduced the fraction of H460 cells from (FIG. 11C), with a concurrent increase in the fraction of T (FIG. 11D). Altogether, these results underscore the ability for S15/CD3 biTE to induce T cell proliferation, activate as well as redirect T cells to kill S15+ cancer cells.

### Example 11

### S15/CD3 Inhibition of Established Human Pancreatic Tumors Correlate with Increased T Cell Activation and Reduced Stromal Barrier

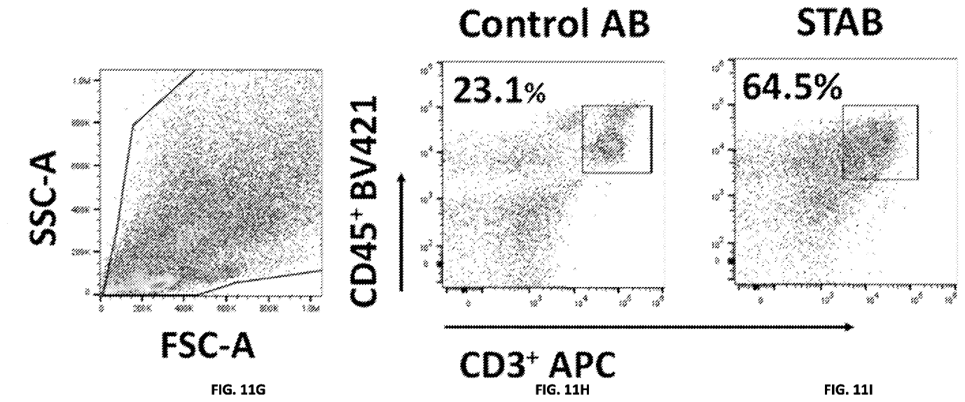

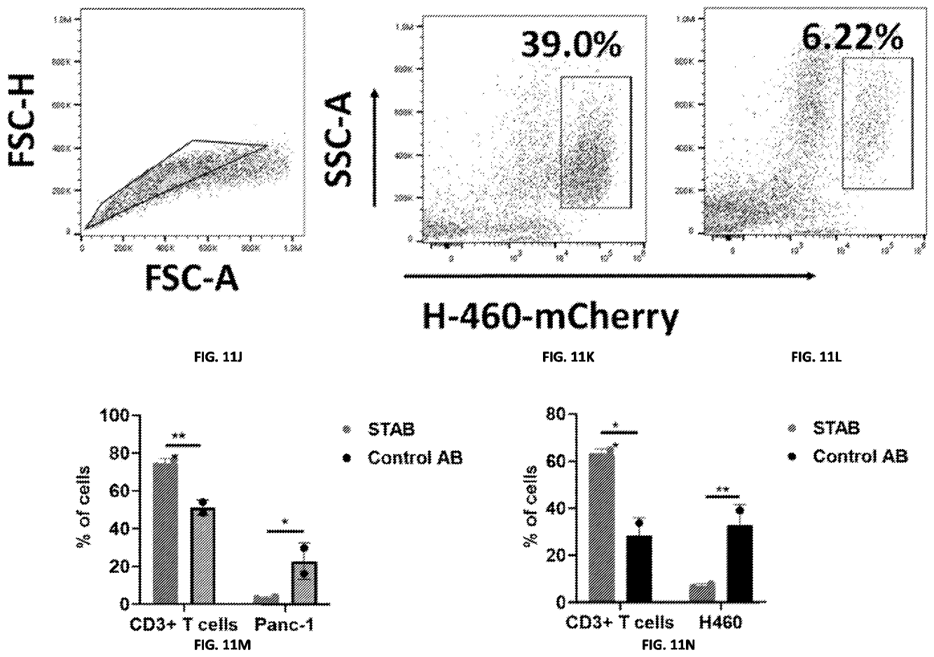

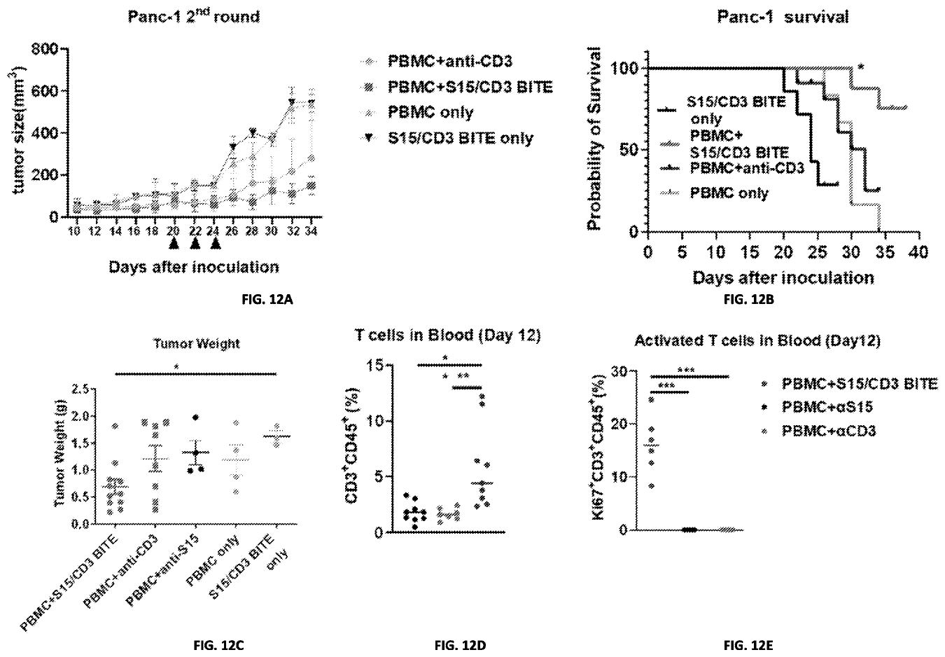

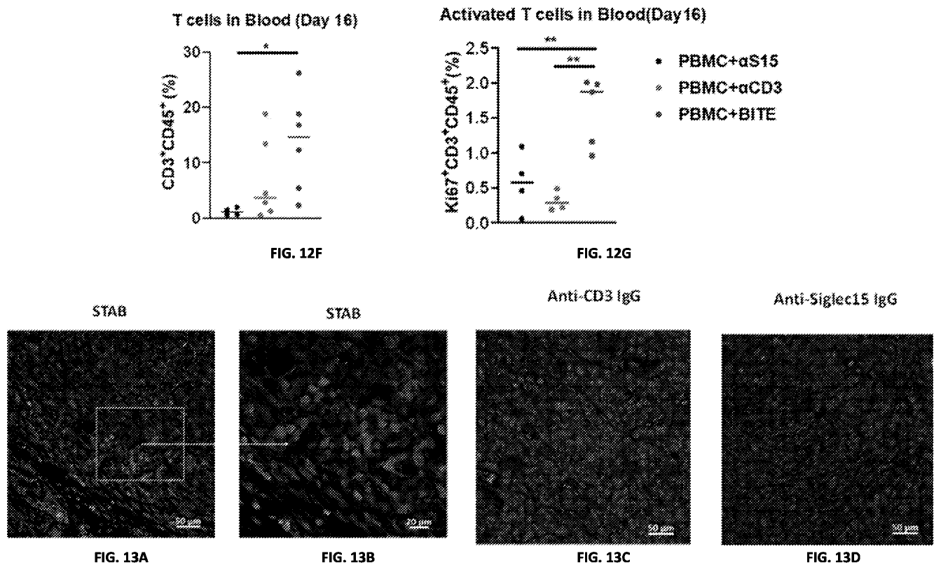

The antitumor efficacy of BiTE was evaluated in a human PDAC model in NSG mice, where human PBMCs from healthy donors and BiTE or control IgGs were infused a total of 3 times over a 5 day period into mice once the tumor volume had reached ~150-200 mm³. During the treatment period, all mice survived, and there was no significant difference in body weight between BiTE treatment group vs. control IgGs. BiTE consistently and effectively inhibited the growth of PDAC tumors and the suppression of tumor growth directly prolonged the survival of tumor bearing mice treated with BiTE (FIGS. 12A and 12B), in sharp contrast to mice treated with control IgGs. This is confirmed by the difference in tumor weights and tumor size at the study endpoint (FIG. 12C). Blood collected from the animals on Days 12 and 16 post tumor-inoculation was also analyzed. In good agreement with the in vitro findings, by Day 12, BiTE appreciably increased the fraction of circulated (CD45+) CD3+ T cells by ~7-fold and ~4-fold over those in mice given anti-S15 IgG and anti-CD3 IgG, respectively (FIG. 12D). Importantly, biTE treatment increased the fraction of activated T cells by ~3.3-fold and ~4.3-fold over those in mice given anti-S15 IgG and anti-CD3 IgG, respectively (FIG. 12E). This trend continued through Day 16, the abundance of T cells in STAB-treated mice remained ~2 fold and ~12-fold greater than those in mice given anti-S15 IgG and anti-CD3 IgG, respectively (FIG. 12F). The abundance of activated T cells detected were appreciably lower than what was measured on Day 12, but remained ~2.83-fold and ~5-fold over those in mice given anti-S15 IgG and anti-CD3 IgG, respectively (FIG. 12G).

### Example 12

### S15/CD3 biTE Promotes Greater T-Cell Distribution and Reduced Tumor-Associated Fibroblasts in Tumors

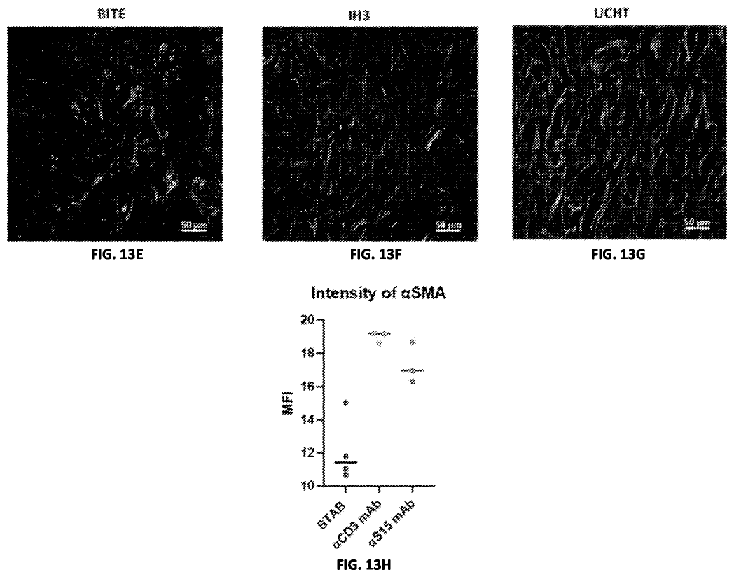

Given the increase in abundance of T cells in the circulation, including activated T cells, whether biTE also enhanced greater T cell distribution to and retention in the tumors was assessed. Immunohistochemistry was performed on tumor sections, and appreciably greater abundance of CD3+ T cells infiltrate within the tumors of mice treated with biTE compare with control groups (FIGS. 13A-13D). These results suggest the anti-tumor activity afforded by biTE is likely directly related to enhanced intratumoral and systemic T cell activity. A hallmark is the abundance of tumor-associated fibroblasts (TAFs). TAFs not only can serve as a source of pro-tumorigenic signals, but also create a stromal barrier that strongly limits the tumoral distribution of various therapeutics. Interestingly, biTE appears to markedly reduce the stroma barrier present in the PDAC tumors (FIGS. 13E-13H), which may in turn enhance the distribution of circulating immune cells as well as biTE into the tumor. The reduced TAF may be caused by biTE redirection of circulating T cells to TAF, which is consistent with unpublished observations that TAF also harbors elevated levels of S15.

### Example 13

### S15/CD3 biTE Inhibits NSCLC and In Vivo and Prolongs T Cell Circulation

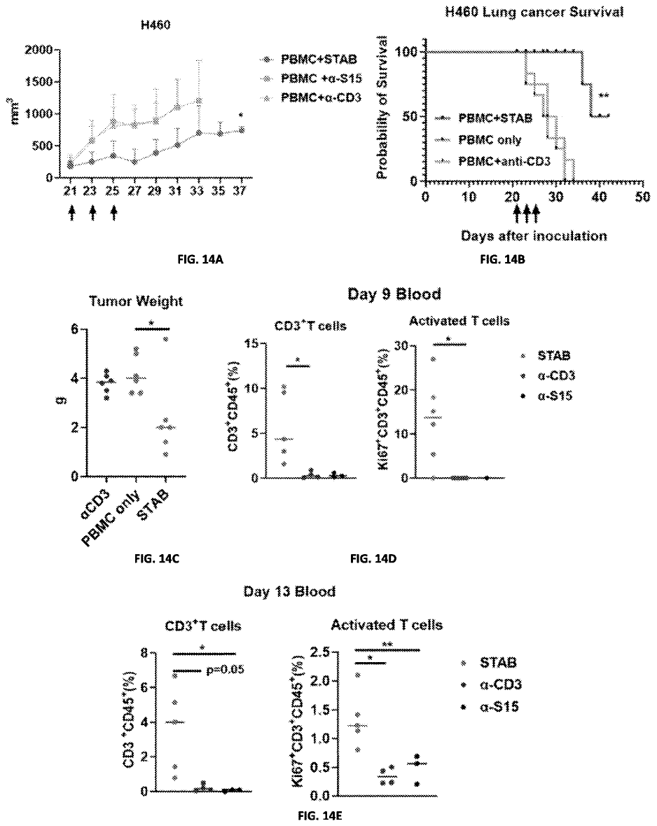

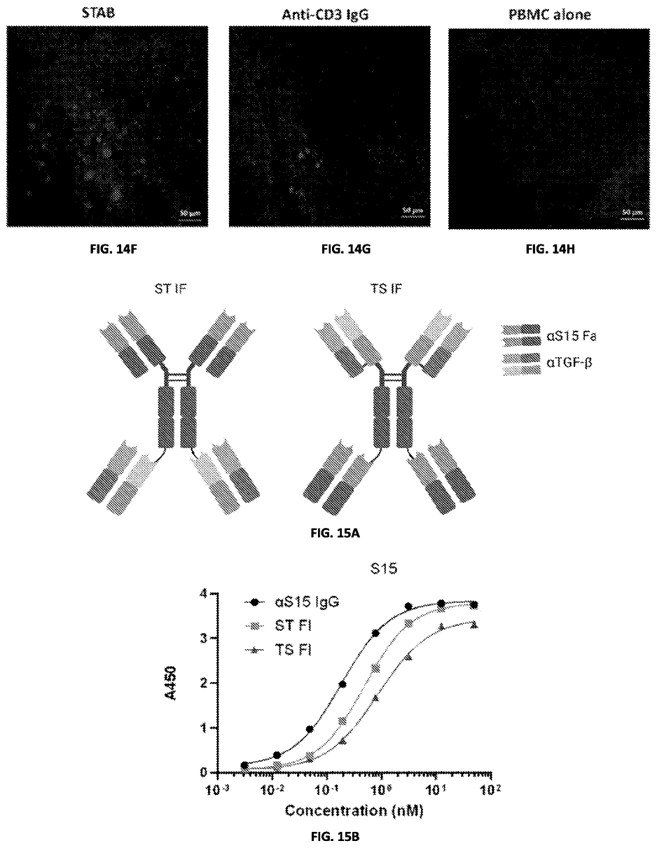

The efficacy of similar biTE treatment was evaluated in H460 NSCLC model in NSG mice. Similar to the findings in mice with PDAC tumors, biTE treatment again strongly limited the growth of H460 tumor over time (FIG. 14A), which in turn greatly prolonged the overall survival of tumor bearing mice (FIG. 14B). The tumor weights in biTE-treated group were significantly lower than the control groups (FIG. 6C). The anti-tumor activity observed correlates with elevated levels of number of circulating CD3⁻ T and activated (Ki67⁻) CD3⁻ T cells as well in the blood (FIGS. 14D and 14E). Increased T cell infiltration was found in the biTE treatment group (FIG. 14G) compared with groups treated with control mAbs (FIGS. 14F-14H).

### Example 14

### Characterization of Binding Affinity of “ST IF” Vs “TS IF”

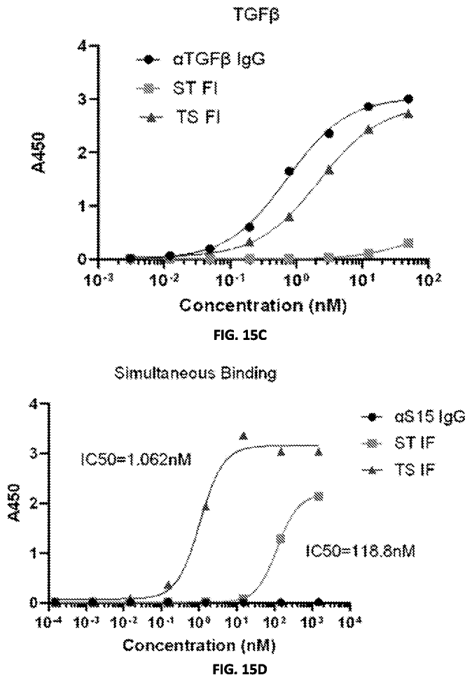

In the bispecifc IgG-Fab (“IF”) format, each Fab can be placed either at the N-terminus of the Fc domain or the C-terminus of the Fc domain. Placement at the C-terminus of the Fc differs requires fusion of the Fc and linker to the N-terminus of the Fab. As many Fabs engage their target antigen at the N-terminus, this has the potential to impact the affinity of the Fab. To investigate the tolerance of the S15 and TGFβ Fabs to their overall placement, two formats were made in which the positions of the Fabs were swapped. These are called ST IF or TS IF, reflective of the relative position of the Fabs within the construct (FIG. 15A). Their binding affinity was compared to the parent IgG by ELISA against S15 (FIG. 15B) and TGFβ (FIG. 15C). The affinity against S15 was similar to the IgG in both constructs. While TS IF did experience a drop compared to ST IF, both remained in the single digit nanomolar affinity range. In contrast, binding to TGFβ was impacted by position. TS IF retained comparable affinity to the parent IgG, while ST IF was decreased by more than an order of magnitude. This led to an overall improvement of the simultaneous binding by nearly 100-fold in TS IF compared to ST IF (FIG. 15D).

### Example 15

### TS IF Increases Survival Relative to ST IF in Mice with Established 4T1 TNBC

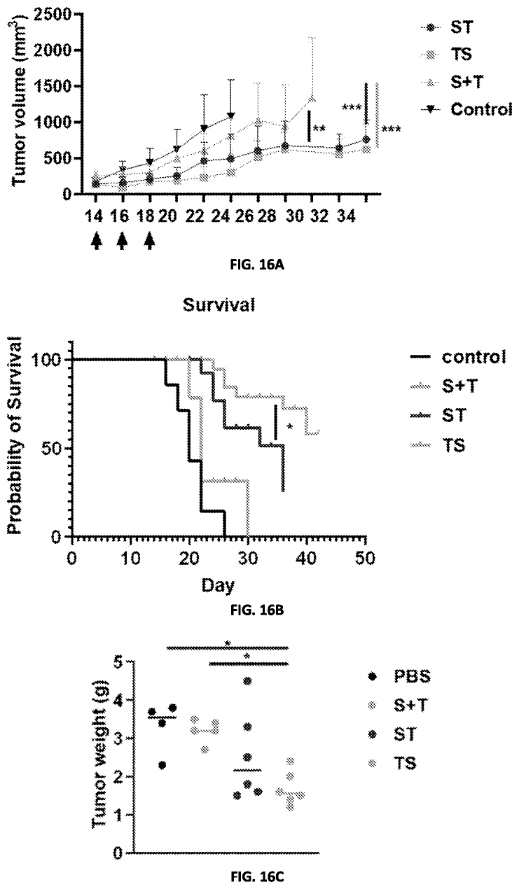

The relative efficacy of TS IF therapy compared to ST IF therapy was assessed in 4T1 triple negative breast cancer murine model. Treatments were initiated on Day 14 after inoculation with 4T1 tumors, and administered a total of 3 times delivered at 2 day intervals. The treatments included the TS IF, ST IF, a cocktail of a mAb against S15 and a mAb against TGF-β, and a saline only control. Clear evidence of tumor suppression was observed with all three Ab treatment conditions, with anti-tumor activity apparent following the very first of the three doses (FIG. 16A). Both ST IF and TS IF showed higher effective inhibition of tumor growth compared to either the cocktail or PBS control (FIGS. 16A, 16C). While tumor growth trended lower in TS IF compared to ST IF, there was minimal difference in tumor growth. However, overall survival was significantly improved in mice treated with TS IF rather than ST IF. Half of mice receiving TS IF survived more than 40 days (FIG. 16B), after all other mice had reached the point of humane sacrifice.

### Example 16

### TS IF Improves Modulation of Immune Cell Presence and Activation within the Tumor Compared to ST IF

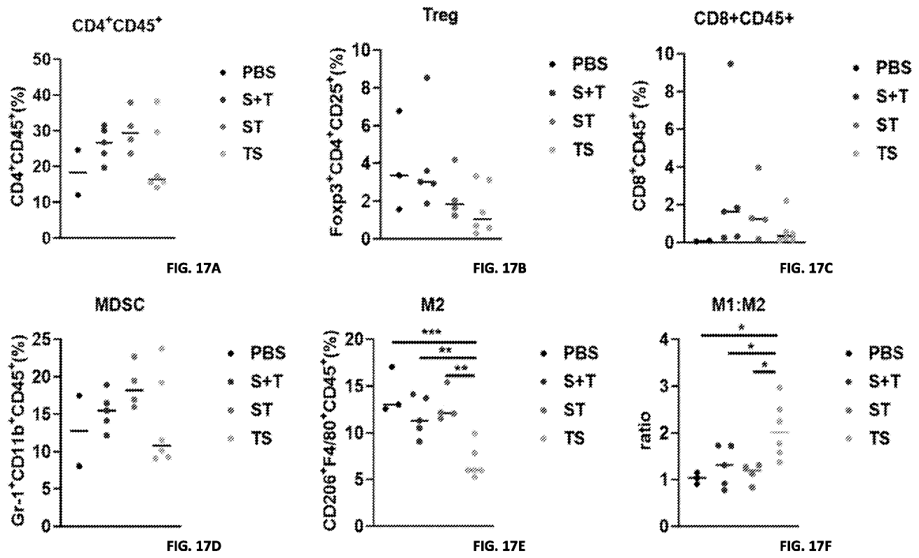

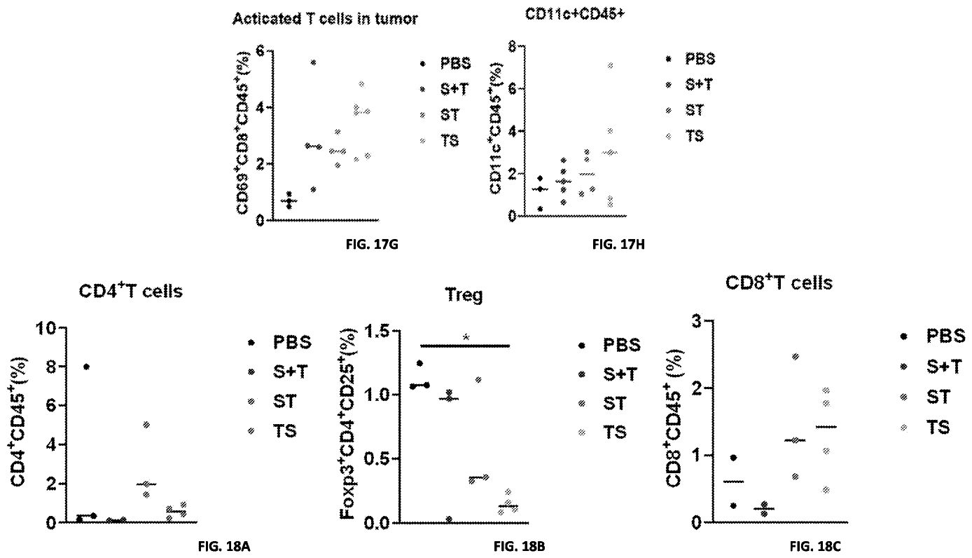

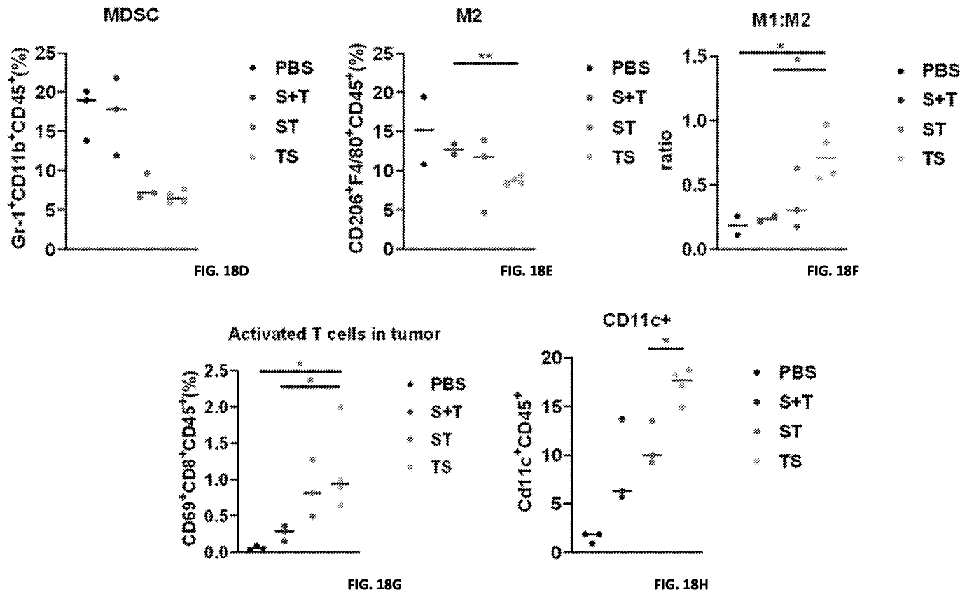

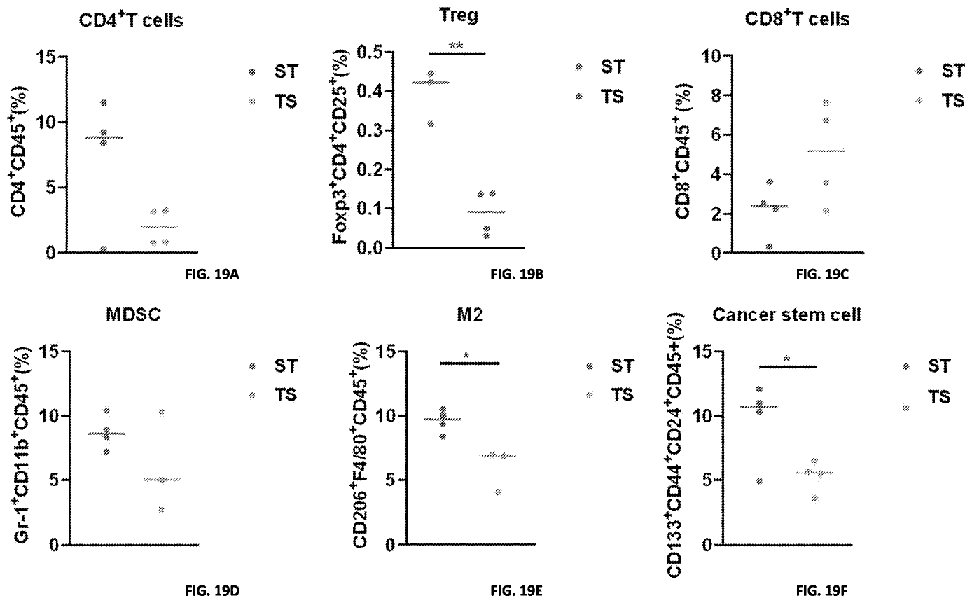

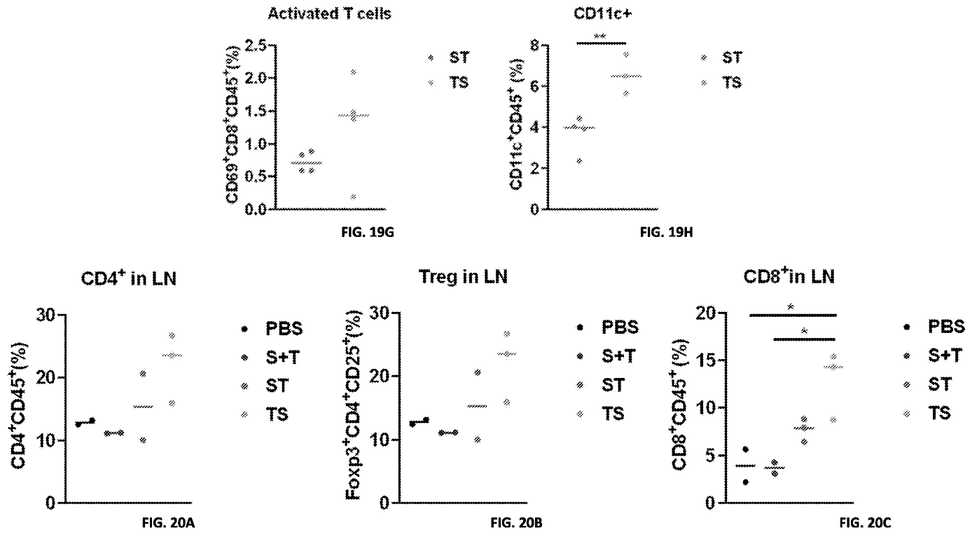

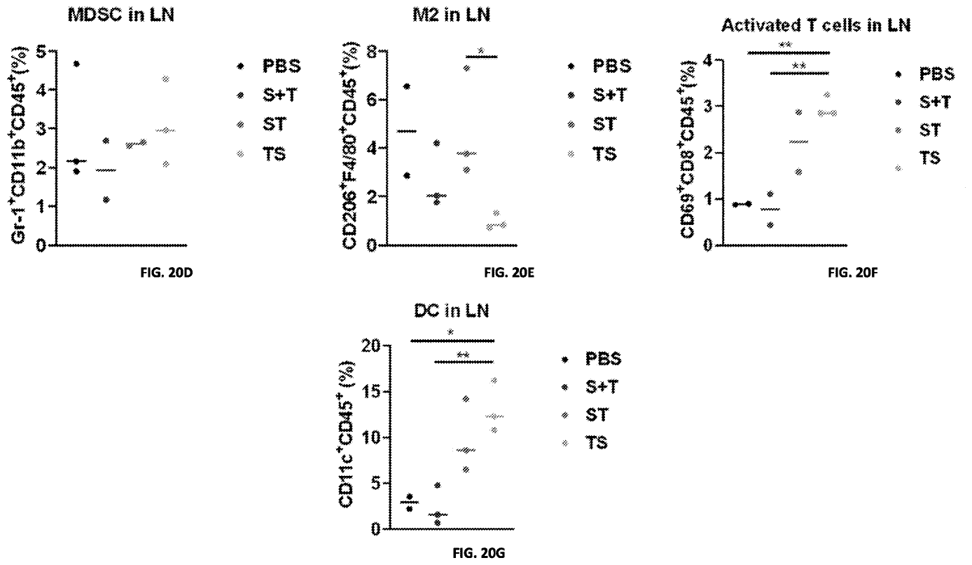

To assess how modulation of the tumor microenvironment may contribute to differences in progression and survival seen between the two formats of ST bsAb, immune cell infiltration was evaluated at various time points in the study. At days 7 (FIGS. 17A-17H), 14 (FIGS. 18A-18H) and 20 (FIGS. 20A-20AH) post-final treatment, subsets of mice were sacrificed, tumors homogenized, and stained for various immune cell populations evaluated by flow cytometry. On day 7, both TS IF and ST IF showed similar amounts of T cell infiltration and activation (FIGS. 17A-17C, 17G), as well as similar levels of dendritic cells (FIG. 17H). Myeloid-derived suppressor cells (MDSCs) trended downward in the TS IF group compared to ST IF, but did not achieve statistical significance (FIG. 17D). Notably, the TS IF treatment group had lower levels M2 macrophages and a more favorable overall ratio of M1-to-M2 macrophages compared to ST IF, consistent with a more immunologically active microenvironment. On day 14, trends in macrophage populations remain similar to day 7, and TS IF additionally exhibits statistically significant reduction in regulatory T-cells compared to control conditions (FIG. 18B). Both TS IF and ST IF have reached statistically significant increases in T cell activation compared to control (FIG. 18G), and TS IF also exhibits increased levels of dendritic cells compared to ST IF (FIG. 18H). By day 20 post final treatment, only mice in ST IF and TS IF treatment groups remained. TS IF sustains lower levels of regulatory T cells (FIG. 19B), immunosuppressive M2 macrophages (FIG. 19E), and cancer stem cells (FIG. 19F) as well as higher levels of dendritic cells (FIG. 19H) relative to ST IF even at the study end point. Taken together, this data indicates TS IF is inducing a more immune-reactive tumor microenvironment compared to ST IF, and that this is sustained better over time.

### Example 17

### TS IF and ST IF Promote Systemic Immune Activation

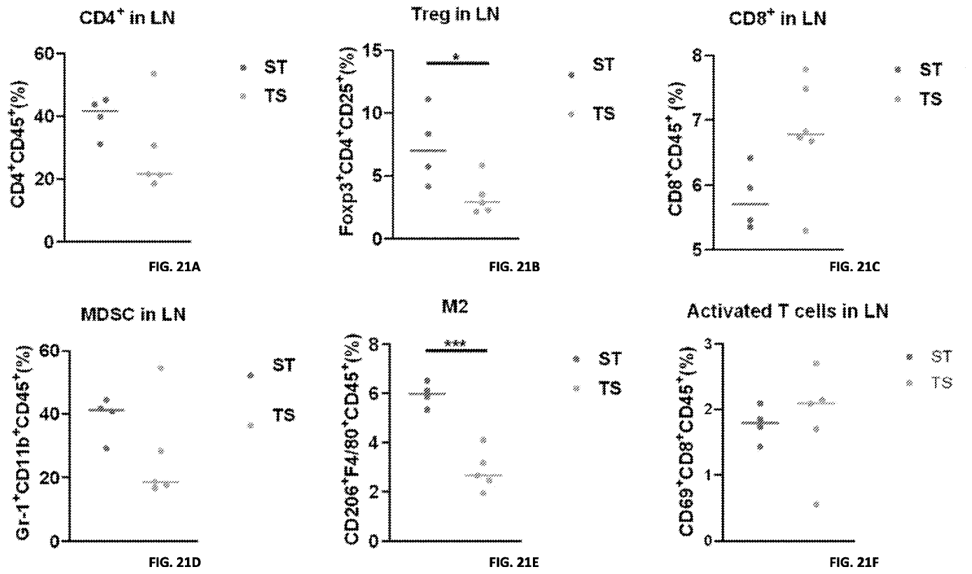

As systemic immune response is also associated with improved patient prognosis, how TS IF and ST IF modulated circulating immune cells was assessed. At days 14 (FIGS. 20A-20G) and 20 (FIGS. 21A-21G) post-final treatment, lymph nodes from a subset of mice were homogenized and assessed by flow cytometry for various immune cell populations. At day 14, both ST IF and TS IF exhibited increased levels of circulating CD8+ T cells (FIG. 20C), activated T cells (FIG. 20F) and dendritic cells (FIG. 20G) relative to control conditions. Additionally, TS IF had decreased levels of circulating M2 macrophages (FIG. 20E). By day 20, TS IF also exhibited statistically significantly reduced circulating regulatory T cells and M2 macrophages compared to ST IF (FIGS. 21B, 21E). Overall, systemic modulation of immune activation between TS IF and ST IF were similar. The main systemic effect of TS IF compared to ST IF was a reduction in circulating immunosuppressive cells such as regulatory T cells and M2 macrophages, which was broadly reflective of the differences within the tumor microenvironment.

### Example 18

### TS IF Improves Clearance of TGFβ within the TME Compared to ST IF

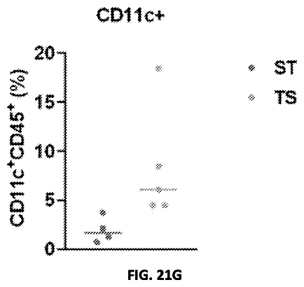

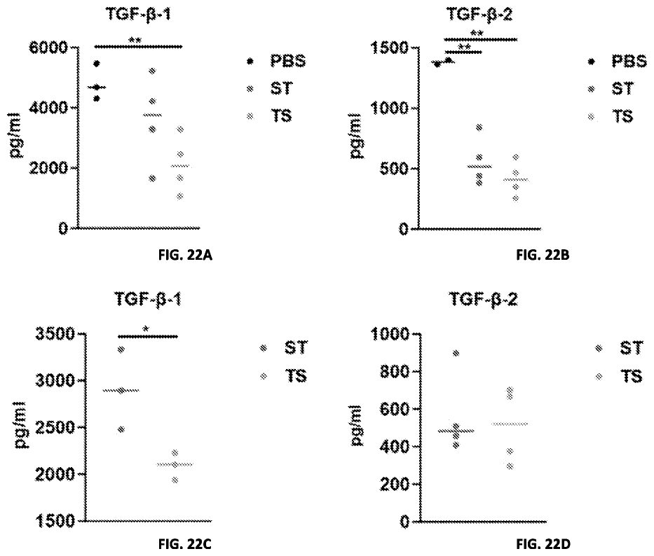

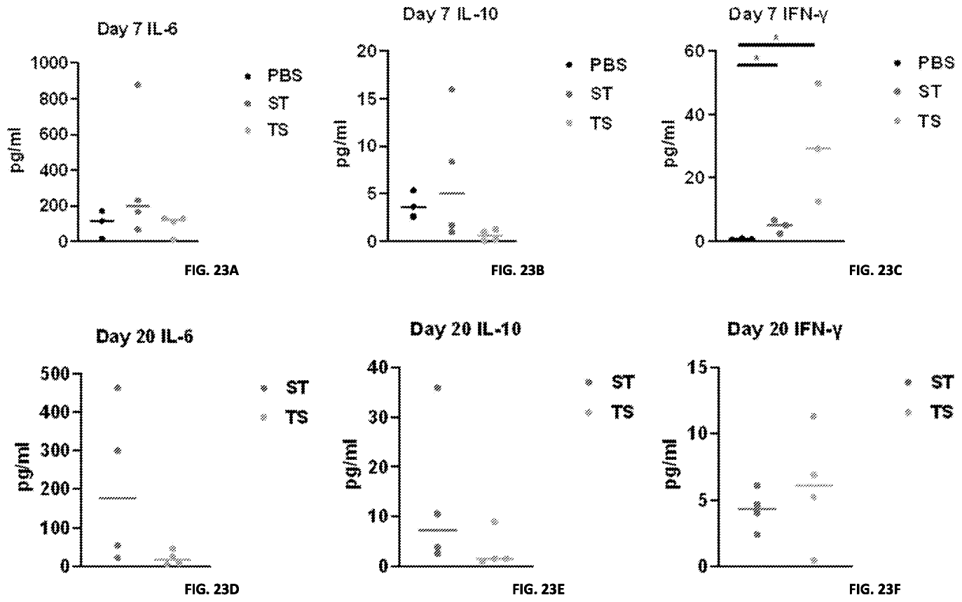

As capture of TGFβ from the TME is the proposed mechanism by which ST bsAbs may improve immunological activity within the tumor, TGFβ31 and TGFβ2 levels were assessed within the tumor on day 7 (FIGS. 22A-22B) and day 20 (FIGS. 22C-22D) post final treatment via luminex assay of homogenized tumor samples. On day 7, both ST IF and TS IF exhibit lower TGF p31 (FIG. 22A) and TGF p2 (FIG. 22B) compared to saline control. On day 20, levels remain similar to day 7 in both conditions, and TS IF treated samples had statistically significantly lower TGF p1 levels compared to ST IF (FIG. 22C). Levels of TGF p2 were similar between both groups (FIG. 22D). The results indicate that TS IF reduces TGFβ concentration relative to ST IF primarily through capture of TGFβ1. Intratumoral levels of cytokines of IL-6, IL-10, and IFNγ were also assessed on days 7 (FIGS. 23A-23C) and 20 (FIGS. 23D-23F) post final treatment. Generally, concentrations of these cytokines were similar between ST IF, TS IF, and control conditions, with the exception of IFNγ levels on day 7 (FIG. 23C). Levels of IFNγ were increased in TS IF treated samples both compared to control and ST IF treated samples on day 7, however this increase was no longer apparent by day 20 (FIG. 23F).

Siglec-15 IgG-CD3 svfc Heavy Chain (Siglec-15IgG-CD3scfv-HC)
(SEQ ID NO: 4): Siglec 15 VH (SEQ ID NO: 3) bolded; anti-CD3
scfv (SEQ ID NO: 5) underlined
QVQLQESGPGLVKPSETLSLTCTVSGFSLSNYGVHWVRQPPGKGLEWIVLIWSDGSTTY
NSALKSRVTISKDTSKNQVSLKLSSVTAADTAVYYCARHPYDDYSGYYYTMDYWGQGTL
VTVSSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSREEMTKNQV
SLTCLVKGFYPSDIAVEWESNGQPENNYKTTPPVLDSDGSFFLYSKLTVDKSRWQQGNVFSCS
VMHEALHNHYTQKSLSLSPGKGGGGSGGGGSGGGGSGGGGSGGGGSGGGGSDIQMTQSPSS
LSASVGDRVTITCRASQDIRNYLNWYQQKPGKAPKLLIYYTSRLESGVPSRFSGSGSGTDYTLT
ISSLQPEDFATYYCQQGNTLPWTFGQGTKVEIKGGGGSGGGGSGGGGSEVQLVESGGGLVQP
GGSLRLSCAASGYSFTGYTMNWVRQAPGKGLEWVALINPYKGVSTYNQKFKDRFTISVDKSK
NTAYLQMNSLRAEDTAVYYCARSGYYGDSDWYFDVWGQGTLVTVSS
Siglec-15 LC
DIQMTQSPSSLSASVGDRVTITCKASDHINNWLAWYQRKPGKAPKLLISGATSLETGVPSRFSG
SGSGTDYTFTISSLQPEDIATYYCQQYWSSPLTFGGGTKVEIKRTVAAPSVFIFPPSDEQLKSGT
ASVVCLLNNFYPREAKVQWKVDNALQSGNSQESVTEQDSKDSTYSLSSTLTLSKADYEKHKV
YACEVTHQGLSSPVTKSFNRGEC (SEQ ID NO: 6)
Siglec-15 LC in CD3 bite
DIQMTQSPSSLSASVGDRVTITCKASDHINNWLAWYQQKPGKAPKLLISGATSLETGVPSRFSG
SGSGTDYTFTISSLQPEDIATYYCQQYWSSPLTFGGGTKVEIKRTVAAPSVFIFPPSDEQLKSGT
ASVVCLLNNFYPREAKVQWKVDNALQSGNSQESVTEQDSKDSTYSLSSTLTLSKADYEKHKV
YACEVTHQGLSSPVTKSFNRGEC (SEQ ID NO: 7)
TGFB Light Chain
RIQMTQSPSSLSASVGDRVTITCRASQSVLYSSNQKNYLAWYQDKPGKAPKLLIYWASTRESG
VPSRFSGSGSGTDFTLTISSLQPEDFATYYCHQYLSSDTFGQGTKVEIKRTVAAPSVFIFPPSDK
QLKSGTARVVCLLNNFYPREAKVQWKVDNALQSGNSQESVTEQDSKDSTYSLSSTLTLSKAD
YEKHKVYACEVTHQGLSSPVTKSFNRGEC (SEQ ID NO: 8)
ST IF (Siglec15-TGF-β IgG-Fab) - Heavy Chain
QVQLQESGPGLVKPSETLSLTCTVSGFSLSNYGVHWVRYPPGKGLEWIVLIWSDGSTTYNSAL
KSRVTISKDTSKNQVSLKLSSVTAADTAVYYCARHPYDDYSGYYYTMDYWGQGTLVTVSSA
STKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCSVMHEA
LHNHYTQKSLSLSPGKGGGGSGGGGSGGGGSGGGGSGGGGSGGGGSEVQLVESGGGLVQPG
GSLRLSCAASGYAFTNYLIEWVRKAPGKGLEWVGVINPGSGGSNYNEKFKGRATISADNSKN
TLYLQMNSLRAEDTAVYYCARSGGFYFDYWGQGTLVTVSSASTKGPSVFPLAPSSKSTSGGT
AALGCLVADYFPEPVTVSWNSGALTSGVHTFPAVLQSSGLYSLSSVVTVPSSSLGTQTYICNV
NHKPSNTKVDEKVEPKSCDKTHT (SEQ ID NO: 9)
TS IF (TGF-β-Siglec15 IgG-Fab) Heavy Chain
EVQLVESGGGLVQPGGSLRLSCAASGYAFTNYLIEWVRKAPGKGLEWVGVINPGSGGSNYNE
KFKGRATISADNSKNTLYLQMNSLRAEDTAVYYCARSGGFYFDYWGQGTLVTVSSASTKGPS
VFPLAPSSKSTSGGTAALGCLVADYFPEPVTVSWNSGALTSGVHTFPAVLQSSGLYSLSSVVT
VPSSSLGTQTYICNVNHKPSNTKVDEKVEPKSCDKTHTCPPCPAPELLGGPSVFLFPPKPKDTL
MISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYT
QKSLSLSPGKGSGGGGSGGGGSGGGGSGGGGSGGGGSGGGGSQVQLQESGPGLVKPSETLSL
TCTVSGFSLSNYGVHWVRYPPGKGLEWIVLIWSDGSTTYNSALKSRVTISKDTSKNQVSLKLS
SVTAADTAVYYCARHPYDDYSGYYYTMDYWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICN
VNHKPSNTKVDKKVEPKSCDKTHT (SEQ ID NO: 10)

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

What is claimed is:

1. A method for treating or delaying the progression of cancer comprising administering to a subject in need thereof an effective amount of a bispecific binding molecule configured to specifically bind:
Siglec15 (S15); and
one of an anti-inflammatory cytokine or chemokine, a tumorigenic cytokine or chemokine, or a T cell activator.

2. The method of claim 1, wherein the anti-inflammatory chemokine or cytokine or tumorigenic chemokine or cytokine is selected from the group consisting of: TGF-β; IL-1 receptor agonist; IL-4; IL-10; IL-11; IL-13; IL-19; IL-20; IL-22; IL-23; IL-24; IL-26; IL-27; IL-28; IL-29; IL-33; IL-35; IL-37; IFN-α; IFN-γ; and CXCL12.

3. The method of claim 2, wherein the anti-inflammatory chemokine or cytokine or tumorigenic chemokine or cytokine is TGF-β.

4. The method of claim 1, wherein the T cell activator is selected from the group consisting of: CD2, CD3, CD4, CD8, and CD28.

5. The method of claim 4, wherein the T cell activator is CD3.

6. The method of any of claims 1-5, wherein the bispecific binding molecule is a bispecific antibody, or fragment thereof, having one or more antigen binding sites directed against S15 and one or more antigen binding sites directed against one of the anti-inflammatory cytokine or chemokine, the tumorigenic cytokine or chemokine, or the T cell activator.

7. The method of claim 6, wherein the bispecific antibody comprises one or more V_{H} domains each comprising an HCDR1, an HCDR2, and an HCDR3, and one or more V_{L} domain each comprising an LCDR1, an LCDR2, and an LCDR3 from an anti-Siglec15 antibody.

8. The method of claim 6 or 7, wherein the bispecific antibody comprises one or more antigen binding sites directed against the anti-inflammatory cytokine or chemokine, the tumorigenic cytokine or chemokine, or the T cell activator.

9. The method of any of claims 6-8, wherein the bispecific antibody comprises one or more V_{H} domains each comprising an HCDR1, an HCDR2, and an HCDR3, and one or more V_{L} domains each comprising an LCDR1, an LCDR2, and an LCDR3 from an antibody directed to an anti-inflammatory cytokine or chemokine, a tumorigenic cytokine or chemokine, or a T cell activator.

10. The method of any of claims 1-9, wherein the method comprises administering two or more bispecific binding molecules.

11. The method of claim 10, wherein a first bispecific binding molecule specifically binds Siglec15 (S15) and an anti-inflammatory cytokine or chemokine, a tumorigenic cytokine or chemokine, and wherein a second bispecific binding molecule specifically binds Siglec15 (S15) and a T cell activator.

12. The method of claim 11, wherein the first bispecific binding molecule specifically binds Siglec15 (S15) and TGF-β.

13. The method of claim 11 or 12, wherein the second bispecific binding molecule specifically binds Siglec15 (S15) and CD3.

14. The method of any of claims 1-13, wherein the cancer is breast cancer, liver cancer, lung cancer, or pancreatic cancer.

15. The method of any of claims 1-14, wherein the cancer is triple negative breast cancer.

16. The method of any of claims 1-14, wherein the cancer is pancreatic cancer.

17. The method of any of claims 1-16, wherein the administering reduces or clears the tumor burden in the subject, decreases or prevents metastasis, increases survival, or a combination thereof.

18. The method of claim 17, wherein administration of the bispecific binding molecule has increased effectiveness compared to methods comprising administration of an antibody against the cancer cell membrane-associated target and an antibody against the anti-inflammatory cytokine or chemokine, the tumorigenic cytokine or chemokine, or the T cell activator alone or a combination thereof.

19. The method of any of claims 1-18, wherein the administering activates T cells.

20. The method of any of claims 1-19, further comprising administering one or more chemotherapeutic agents to the subject, administering radiation therapy to the subject, surgical resection of cancerous tissue or tumors, or a combination thereof.

21. A therapeutically effective amount of a composition comprising a bispecific binding molecule configured to specifically bind: Siglec15 (S15); and one of an anti-inflammatory cytokine or chemokine, a tumorigenic cytokine or chemokine, or a T cell activator for use in treating or delaying the progression of cancer in a subject.

22. The composition for use of claim 21, wherein the anti-inflammatory chemokine or cytokine or tumorigenic chemokine or cytokine is selected from the group consisting of: TGF-β; IL-1 receptor agonist; IL-4; IL-10; IL-11; IL-13; IL-19; IL-20; IL-22; IL-23; IL-24; IL-26; IL-27; IL-28; IL-29; IL-33; IL-35; IL-37; IFN-α; IFN-γ; and CXCL12.

23. The composition for use of claim 22, wherein the anti-inflammatory chemokine or cytokine or tumorigenic chemokine or cytokine is TGF-β.

24. The composition for use of claim 21, wherein the T cell activator is selected from the group consisting of: CD2, CD3, CD4, CD8, and CD28.

25. The composition for use of claim 24, wherein the T cell activator is CD3.

26. The composition for use of any of claims 21-25, wherein the bispecific binding molecule is a bispecific antibody, or fragment thereof, having one or more antigen binding sites directed against S15 and one or more antigen binding sites directed against one of the anti-inflammatory cytokine or chemokine, the tumorigenic cytokine or chemokine, or the T cell activator.

27. The composition for use of claim 26, wherein the bispecific antibody comprises one or more V_{H} domains each comprising an HCDR1, an HCDR2, and an HCDR3, and one or more V_{L} domains each comprising an LCDR1, an LCDR2, and an LCDR3 from an anti-Siglec15 antibody.

28. The composition for use of claim 26 or 27, wherein the bispecific antibody comprises one or more antigen binding sites directed against the anti-inflammatory cytokine or chemokine, the tumorigenic cytokine or chemokine, or the T cell activator.

29. The composition for use of any of claims 21-28, further comprising one or more chemotherapeutic agents.

30. The composition for use of any of claims 21-29, wherein the subject has breast cancer, liver cancer, lung cancer, or pancreatic cancer.

31. A kit comprising a medicament comprising:
a bispecific binding molecule configured to specifically bind: Siglec15 (S15); and one of an anti-inflammatory cytokine or chemokine, a tumorigenic cytokine or chemokine, or a T cell activator; and
instructions for administration of the medicament for treating or delaying progression of cancer in an individual.

32. The kit of claim 31, further comprising one or more chemotherapeutic agents.


SEQUENCE LISTING (10 sequences)

SEQ ID NO: 1 - protein, 30 aa, synthetic construct
GGGGSGGGGSGGGGSGGGGSGGGGSGGGGS

SEQ ID NO: 2 - protein, 107 aa, synthetic construct
DIQMTQSPSSLSASVGDRVTITCKASDHINNWLAWYQQKPGKAPKLLISGATSLETGVPSRFSGSGSGTDYTFTISSLQPEDIATYYCQQYWSSPLTFGGGTKVEIK

SEQ ID NO: 3 - protein, 123 aa, synthetic construct
QVQLQESGPGLVKPSETLSLTCTVSGFSLSNYGVHWVRQPPGKGLEWIVLIWSDGSTTYNSALKSRVTISKDTSKNQVSLKLSSVTAADTAVYYCARHPYDDYSGYYYTMDYWGQGTLVTVSS

SEQ ID NO: 4 - protein, 727 aa, synthetic construct
QVQLQESGPGLVKPSETLSLTCTVSGFSLSNYGVHWVRQPPGKGLEWIVLIWSDGSTTYNSALKSRVTISKDTSKNQVSLKLSSVTAADTAVYYCARHPYDDYSGYYYTMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 5 - protein, 244 aa, synthetic construct
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 6 - protein, 214 aa, synthetic construct
DIQMTQSPSSLSASVGDRVTITCKASDHINNWLAWYQRKPGKAPKLLISGATSLETGVPSRFSGSGSGTDYTFTISSLQPEDIATYYCQQYWSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 214 aa, synthetic construct
DIQMTQSPSSLSASVGDRVTITCKASDHINNWLAWYQQKPGKAPKLLISGATSLETGVPSRFSGSGSGTDYTFTISSLQPEDIATYYCQQYWSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 219 aa, synthetic construct
RIQMTQSPSSLSASVGDRVTITCRASQSVLYSSNQKNYLAWYQDKPGKAPKLLIYWASTRESGVPSRFSGSGSGTDFTLTISSLQPEDFATYYCHQYLSSDTFGQGTKVEIKRTVAAPSVFIFPPSDKQLKSGTARVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 708 aa, synthetic construct
QVQLQESGPGLVKPSETLSLTCTVSGFSLSNYGVHWVRYPPGKGLEWIVLIWSDGSTTYNSALKSRVTISKDTSKNQVSLKLSSVTAADTAVYYCARHPYDDYSGYYYTMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSEVQLVESGGGLVQPGGSLRLSCAASGYAFTNYLIEWVRKAPGKGLEWVGVINPGSGGSNYNEKFKGRATISADNSKNTLYLQMNSLRAEDTAVYYCARSGGFYFDYWGQGTLVTVSSASTKGPSVFPLAPSSKSTSGGTAALGCLVADYFPEPVTVSWNSGALTSGVHTFPAVLQSSGLYSLSSVVTVPSSSLGTQTYICNVNHKPSNTKVDEKVEPKSCDKTHT

SEQ ID NO: 10 - protein, 710 aa, synthetic construct
EVQLVESGGGLVQPGGSLRLSCAASGYAFTNYLIEWVRKAPGKGLEWVGVINPGSGGSNYNEKFKGRATISADNSKNTLYLQMNSLRAEDTAVYYCARSGGFYFDYWGQGTLVTVSSASTKGPSVFPLAPSSKSTSGGTAALGCLVADYFPEPVTVSWNSGALTSGVHTFPAVLQSSGLYSLSSVVTVPSSSLGTQTYICNVNHKPSNTKVDEKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGGGSGGGGSGGGGSGGGGSGGGGSGGGGSQVQLQESGPGLVKPSETLSLTCTVSGFSLSNYGVHWVRYPPGKGLEWIVLIWSDGSTTYNSALKSRVTISKDTSKNQVSLKLSSVTAADTAVYYCARHPYDDYSGYYYTMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT